(19) United States
(12) Patent Application Publication
(10) Pub. No.: US 2023/0270720 A1
(43) Pub. Date: Aug. 31, 2023

(54) MAVOGLURANT, A MGLUR5 ANTAGONIST, FOR USE IN THE TREATMENT IN THE REDUCTION OF OPIOID USE

(71) Applicant: Novartis AG, Basel (US)

(72) Inventors: Richard Carl Elciario DOLMETSCH, Concord, MA (US); Fabrizio GASPARINI, Basel (CH); Baltazar GOMEZ-MANCILLA, Rosenau (FR)

(21) Appl. No.: 18/005,364

(22) PCT Filed: Jul. 15, 2021
(86) PCT No.: PCT/IB2021/056397

§ 371 Date: Jan. 13, 2023

Related U.S. Application Data
(60) Provisional application No. 63/124,537, filed on Dec. 11, 2020.
(60) Provisional application No. 63/053,090, filed on Jul. 17, 2020.

(51) Int. Cl. A61K 31/404; A61P 25/36

(52) U.S. Cl. CPC A61K 31/404; A61P 25/36

(57) ABSTRACT

The invention relates to the use of mavoglurant, or a pharmaceutically acceptable salt thereof: in the reduction of opioid use by an opioid use disorder patient; in preventing relapse into opioid use by an opioid use disorder patient; in the promotion of opioid abstinence by an opioid use disorder patient; in the treatment of the symptoms of depression or anxiety associated with opioid use disorder.

The present invention relates to uses of a mGluR5 antagonist.

### FIELD OF THE INVENTION

The invention relates to the use of the mGluR5 antagonist named mavoglurant, or a pharmaceutically acceptable salt thereof, in the reduction of opioid use by an opioid use disorder patient; in preventing relapse into opioid use by an opioid use disorder patient; in the promotion of opioid abstinence by an opioid use disorder patient; in the treatment of the symptoms of depression or anxiety associated with opioid use disorder. In particular, it relates to the use of mavoglurant, or a pharmaceutically acceptable salt thereof, in the reduction of opioid use/in preventing relapse into opioid use, by an opioid use disorder patient.

### BACKGROUND OF THE INVENTION

Opioid use disorder is a complex psychiatric disorder (e.g. defined with reference to DSM-5 criteria; i.e. according to the Diagnostic and Statistical Manual of Mental Disorders. 5th Edition, Washington, D.C.: American Psychiatric Association, 2013), which continues to grow into a significant worldwide health problem having adverse medical, social and economic effects (Bahorilk et al J Addict Med. 2017; 11(1): 3-9). Socio-economic dangers associated with opioid use include increased crime, violence and overdose leading to death.

The mGlu5 receptor (mGluR5) has been implicated in the rewarding effect of various drugs of abuse and drug-seeking behaviour. Van der Kam et al. showed that the mGluR5 antagonist 2-Methyl-6-(phenylethynyl)-pyridine (MPEP) dose dependently reduced (75%) the self-administration (SA) of ketamine and modestly (20%) reduced the SA of heroin in a rat model of drug dependence (Van der Kam et al Behavioural Pharmacology 18 (2007) 717-724). Brown et al. investigated the impact of antagonism of mGluR5 with the selective negative allosteric modulator 3-[(2-methyl-1,3-thiazol-4-yl)ethynyl]pyridine (MTEP) on operant self-administration of morphine as well as cue-induced drug-seeking in adult CD1 mice. Administration of MTEP (20 mg/kg, i.p.) attenuated operant responding for morphine (0.1 mg/kg/infusion) and cue-induced morphine-seeking after a period of forced abstinence.

### SUMMARY OF THE INVENTION

The invention relates to the use of mavoglurant, or a pharmaceutically acceptable salt thereof:
in the reduction of opioid use by an opioid use disorder patient;
in preventing relapse into opioid use by an opioid use disorder patient;
in the promotion of opioid abstinence by an opioid use disorder patient;
in the treatment of the symptoms of depression or anxiety associated with opioid use disorder.

### BRIEF DESCRIPTION OF DRAWINGS

FIG. 1: Effects of Compound (III) on foot licking latency

FIG. 2: Effects of Compound (I) on foot licking latency

FIG. 3: Effect of Mavoglurant (3, 10, 30 mg/kg), MPEP (20 mg/kg) and Buprenorphine (0.2 mg/kg) on the Remifentanil self-administration. A) Effect on the number of self-administered infusions per session of Remifentanil. B) Effect on the rate of lever press (speed of lever pressing reported as responses/s/session).

FIG. 4A: Effect of Mavoglurant on the cue-induced reinstatement of Remifentanil. Mavoglurant at 10 mg/kg.

FIG. 4B: Effect of Mavoglurant on the cue-induced reinstatement of Remifentanil. Mavoglurant at 3 mg/kg.

FIG. 4C: Effect of MPEP on the cue-induced reinstatement of Remifentanil. MPEP at 20 mg/kg.

### DETAILED DESCRIPTION OF THE INVENTION

Mavoglurant may be an ideal candidate for treating patients diagnosed with opioid use disorder, having therapeutic advantages for said patient population, such as one or more of the following:
i) promoting opioid abstinence, for example, compared to placebo, for example by maintaining abstinence or by reducing the amount or frequency of opioid use, for example as assessed by urinalysis (e.g. by measuring metabolites of opioids in urine) or as assessed by using self-reported opioid use with standardized tools like the Timeline Follow-Back self-report [Sobell, L. C., Sobell, M. B. (1996) Timeline Followback User's Guide: A Calendar Method for Assessing Alcohol and Drug Use. Addiction Research Foundation, Toronto, Ontario, Canada; J. Anal. Toxicolo., 2002, 26: 393-400];
ii) decreasing relapse into opioid use, for example, compared to placebo, for example it increases the time to relapse or the rates of patient relapse in a treatment program, such as a clinical trial;
iii) alleviating (e.g. by eliminating or by reducing intensity, duration or frequency), for example compared to placebo, one or more of symptoms associated with opioid use disorder selected from:
   a. depressive symptoms, for example as assessed from the Beck's Depression Inventory [Beck, A. T. et al., (1961) An inventory for measuring depression. Archives of General Psychiatry, 4, 561-571; Beck, A. T. et al., (1988) Psychometric properties of the Beck Depression Inventory: Twenty-five years of evaluation. Clinical Psychology Review, 8(1), 77-100]; and
   b. anxiety symptoms, for example as assessed from the State-Trait Anxiety Inventory [Spielberger, C. D. (1989). State-Trait Anxiety Inventory: Bibliography (2^{nd} Ed.). Palo Alto, Calif.: Consulting Psychologists Press; Spielberger, C. D. et al., (1983). Manual for the State-Trait Anxiety Inventory. Palo Alto, Calif.: Consulting Psychologists Press].
iv) increasing retention of patients in treatment, for example, compared to placebo, for example it increases the rates of patient retention in a treatment program, such as a clinical trial (e.g. as measured by patient attendance at scheduled clinic visits and/or time to dropout from clinical protocol);
v) it improves global functioning, for example as assessed from the Clinical Global Impression Scale-Severity (CGI-S) and Improvement (CGI-I) (Psychiatry, 2007, 4(7): 28-37); or
vi) it has a favorable therapeutic profile, such as a favorable safety profile or metabolic profile, for example a favorable profile in relation to psychiatric adverse events, genotoxicity, or cardiovascular adverse events (e.g. blood pressure, heart rate, electrocardiography parameters); for example, it has better therapeutic profile (e.g. fewer side effects, decreased off-target effects or decreased toxicity, such as decreased genotoxicity) compared to known therapeutic agent/s that have been used in the treatment of opioid use disorder.

### Embodiments of the Present Invention are

### Embodiments (a)

1a. Mavoglurant, or a pharmaceutically acceptable salt thereof, for use in the reduction of opioid use by an opioid use disorder patient.
2a. Mavoglurant, or a pharmaceutically acceptable salt thereof, for use in preventing relapse into opioid use by an opioid use disorder patient.
3a. Mavoglurant, or a pharmaceutically acceptable salt thereof, for use in the promotion of opioid abstinence by an opioid use disorder patient.
4a. Mavoglurant, or a pharmaceutically acceptable salt thereof, for use in the treatment of the symptoms of depression or anxiety associated with opioid use disorder.
5a. Mavoglurant, or a pharmaceutically acceptable salt thereof, for use according to any one of embodiments 1a to 4a, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
6a. Mavoglurant, or a pharmaceutically acceptable salt thereof, for use according to any one of embodiments 1a to 5a, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
7a. Mavoglurant, or a pharmaceutically acceptable salt thereof, for use according to any one of embodiments 1a to 6a, wherein the use is combined with standardized psychological treatment, for example, at individual or group level.
8a. Mavoglurant, or a pharmaceutically acceptable salt thereof, for use according to any one of embodiments 1a to 7a, wherein the use is combined with psychosocial or behavioral therapy or combination thereof, in particular contingency management based therapy.
9a. Mavoglurant, or a pharmaceutically acceptable salt thereof, for use according to embodiment 8a, wherein the psychosocial or the behavioral therapy is computer-assisted.
10a. Mavoglurant, or a pharmaceutically acceptable salt thereof, for use according to any one of embodiments 1a to 9a, wherein the use is concomitant with methadone or buprenorphine treatment.
11a. Mavoglurant, or a pharmaceutically acceptable salt thereof, for use according to any one of embodiments 1a to 10a, wherein mavoglurant, or a pharmaceutically acceptable salt thereof, is administered in combination with a further active agent.
12a. Mavoglurant, or a pharmaceutically acceptable salt thereof, for use according to embodiment 11a, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorphine), a NET inhibitor (e.g. desipramine) and a NMDA receptor antagonist (e.g. amantadine); or pharmaceutically acceptable salts thereof.
13a. Mavoglurant, or a pharmaceutically acceptable salt thereof, for use according to any one of embodiments 1a to 12a, wherein the patient has a genetic variation associated with a substance use disorder, such as a genetic variation associated with opioid use disorder.
14a. Mavoglurant, or a pharmaceutically acceptable salt thereof, for use according to any one of embodiments 1a to 13a, wherein mavoglurant, or a pharmaceutically acceptable salt thereof, is administered in an immediate-release form or a modified-release form.
15a. Mavoglurant, or a pharmaceutically acceptable salt thereof, for use according to any one of embodiments 1a to 14a, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
16a. Mavoglurant, or a pharmaceutically acceptable salt thereof, for use according to embodiment 15a, wherein mavoglurant, or a pharmaceutically acceptable salt thereof, is administered with food.
17a. Mavoglurant, or a pharmaceutically acceptable salt thereof, for use according to embodiment 15a or 16a, wherein mavoglurant, or a pharmaceutically acceptable salt thereof, is administered in the morning and in the evening separated by a 12 hour interval.
18a. Mavoglurant, or a pharmaceutically acceptable salt thereof, for use according to any of the preceding embodiments, wherein opioid use disorder is associated with binge drinking.

### Embodiments (b)

1b. A pharmaceutical composition comprising mavoglurant, or a pharmaceutically acceptable salt thereof, and at least one pharmaceutically acceptable excipient, for use in the reduction of opioid use by an opioid use disorder patient.
2b. A pharmaceutical composition comprising mavoglurant, or a pharmaceutically acceptable salt thereof, and at least one pharmaceutically acceptable excipient, for use in preventing relapse into opioid use by an opioid use disorder patient.
3b. A pharmaceutical composition comprising mavoglurant, or a pharmaceutically acceptable salt thereof, and at least one pharmaceutically acceptable excipient, for use in the promotion of opioid abstinence by an opioid use disorder patient.
4b. A pharmaceutical composition comprising mavoglurant, or a pharmaceutically acceptable salt thereof, and at least one pharmaceutically acceptable excipient, for use in the treatment of the symptoms of depression or anxiety associated with opioid use disorder.
5b. A pharmaceutical composition comprising mavoglurant, or a pharmaceutically acceptable salt thereof, and at least one pharmaceutically acceptable excipient, for use according to any one of embodiments 1 b to 4b, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
6b. A pharmaceutical composition comprising mavoglurant, or a pharmaceutically acceptable salt thereof, and at least one pharmaceutically acceptable excipient, for use according to any one of embodiments 1 b to 5b, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
7b. A pharmaceutical composition comprising mavoglurant, or a pharmaceutically acceptable salt thereof, and at least one pharmaceutically acceptable excipient, for use according to any one of embodiments 1 b to 6b, wherein the use is combined with standardized psychological treatment, for example, at individual or group level.
8b. A pharmaceutical composition comprising mavoglurant, or a pharmaceutically acceptable salt thereof, and at least one pharmaceutically acceptable excipient, for use according to any one of embodiments 1 b to 7b, wherein the use is combined with psychosocial or behavioral therapy or combination thereof, in particular contingency management based therapy.
9b. A pharmaceutical composition comprising mavoglurant, or a pharmaceutically acceptable salt thereof, and at least one pharmaceutically acceptable excipient, for use according to embodiment 8b, wherein the psychosocial or the behavioral therapy is computer-assisted.
10b. A pharmaceutical composition comprising mavoglurant, or a pharmaceutically acceptable salt thereof, and at least one pharmaceutically acceptable excipient, for use according to any one of embodiments 1 b to 9b, wherein the use is concomitant with methadone or buprenorphine treatment.
11b. A pharmaceutical composition comprising mavoglurant, or a pharmaceutically acceptable salt thereof, and at least one pharmaceutically acceptable excipient, for use according to any one of embodiments 1 b to 10b, wherein mavoglurant, or a pharmaceutically acceptable salt thereof, is administered in combination with a further active agent.
12b. A pharmaceutical composition comprising mavoglurant, or a pharmaceutically acceptable salt thereof, and at least one pharmaceutically acceptable excipient, for use according to embodiment 11b, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorphine), a NET inhibitor (e.g. desipramine) and a NMDA receptor antagonist (e.g. amantadine); or pharmaceutically acceptable salts thereof.
13b. A pharmaceutical composition comprising mavoglurant, or a pharmaceutically acceptable salt thereof, and at least one pharmaceutically acceptable excipient, for use according to any one of embodiments 1 b to 12b, wherein the patient has a genetic variation associated with a substance use disorder, such as a genetic variation associated with opioid use disorder.
14b. A pharmaceutical composition comprising mavoglurant, or a pharmaceutically acceptable salt thereof, and at least one pharmaceutically acceptable excipient, for use according to any one of embodiments 1 b to 13b, which is an immediate-release form or a modified-release form.
15b. A pharmaceutical composition comprising mavoglurant, or a pharmaceutically acceptable salt thereof, and at least one pharmaceutically acceptable excipient, for use according to any one of embodiments 1 b to 14b, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
16b. A pharmaceutical composition comprising mavoglurant, or a pharmaceutically acceptable salt thereof, and at least one pharmaceutically acceptable excipient, for use according to embodiment 15b, wherein mavoglurant, or a pharmaceutically acceptable salt thereof, is administered with food.
17b. A pharmaceutical composition comprising mavoglurant, or a pharmaceutically acceptable salt thereof, and at least one pharmaceutically acceptable excipient, for use according to embodiment 15b or 16b, wherein mavoglurant, or a pharmaceutically acceptable salt thereof, is administered in the morning and in the evening separated by a 12 hour interval.
18b. A pharmaceutical composition comprising mavoglurant, or a pharmaceutically acceptable salt thereof, and at least one pharmaceutically acceptable excipient, for use according to any of the preceding embodiments, wherein opioid use disorder is associated with binge drinking.

### Embodiments (c)

1c. A pharmaceutical combination comprising mavoglurant, or a pharmaceutically acceptable salt thereof, and at least one further pharmaceutical active ingredient, for use in the reduction of opioid use by an opioid use disorder patient.
2c. A pharmaceutical combination comprising mavoglurant, or a pharmaceutically acceptable salt thereof, and at least one further pharmaceutical active ingredient, for use in preventing relapse into opioid use by an opioid use disorder patient.
3c. A pharmaceutical combination comprising mavoglurant, or a pharmaceutically acceptable salt thereof, and at least one further pharmaceutical active ingredient, for use in the promotion of opioid abstinence by an opioid use disorder patient.
4c. A pharmaceutical combination comprising mavoglurant, or a pharmaceutically acceptable salt thereof, and at least one further pharmaceutical active ingredient, for use in the treatment of the symptoms of depression or anxiety associated with opioid use disorder.
5c. A pharmaceutical combination comprising mavoglurant, or a pharmaceutically acceptable salt thereof, and at least one further pharmaceutical active ingredient, for use according to any one of embodiments 1c to 4c, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
6c. A pharmaceutical combination comprising mavoglurant, or a pharmaceutically acceptable salt thereof, and at least one further pharmaceutical active ingredient, for use according to any one of embodiments 1c to 5c, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
7c. A pharmaceutical combination comprising mavoglurant, or a pharmaceutically acceptable salt thereof, and at least one further pharmaceutical active ingredient, for use according to any one of embodiments 1c to 6c, wherein the use is combined with standardized psychological treatment, for example, at individual or group level.
8c. A pharmaceutical combination comprising mavoglurant, or a pharmaceutically acceptable salt thereof, and at least one further pharmaceutical active ingredient, for use according to any one of embodiments 1c to 7c, wherein the use is combined with psychosocial or behavioral therapy or combination thereof, in particular contingency management based therapy.
9c. A pharmaceutical combination comprising mavoglurant, or a pharmaceutically acceptable salt thereof, and at least one further pharmaceutical active ingredient, for use according to embodiment 8c, wherein the psychosocial or the behavioral therapy is computer-assisted.
10c. A pharmaceutical combination comprising mavoglurant, or a pharmaceutically acceptable salt thereof, and at least one further pharmaceutical active ingredient, for use according to any one of embodiments 1c to 9c, wherein the use is concomitant with methadone or buprenorphine treatment.
11c. A pharmaceutical combination comprising mavoglurant, or a pharmaceutically acceptable salt thereof, and at least one further pharmaceutical active ingredient, for use according to any one of embodiments 1c to 10c, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.
12c. A pharmaceutical combination comprising mavoglurant, or a pharmaceutically acceptable salt thereof, and at least one further pharmaceutical active ingredient, for use according to any one of embodiments 1c to 11c, wherein the patient has a genetic variation associated with a substance use disorder, such as a genetic variation associated with opioid use disorder.
13c. A pharmaceutical combination comprising mavoglurant, or a pharmaceutically acceptable salt thereof, and at least one further pharmaceutical active ingredient, for use according to any one of embodiments 1c to 12c, wherein mavoglurant, or a pharmaceutically acceptable salt thereof, is administered in an immediate-release form or a modified-release form.
14c. A pharmaceutical combination comprising mavoglurant, or a pharmaceutically acceptable salt thereof, and at least one further pharmaceutical active ingredient, for use according to any one of embodiments 1c to 13c, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
15c. A pharmaceutical combination comprising mavoglurant, or a pharmaceutically acceptable salt thereof, and at least one further pharmaceutical active ingredient, for use according to embodiment 14c, wherein mavoglurant, or a pharmaceutically acceptable salt thereof, is administered with food.
16c. A pharmaceutical combination comprising mavoglurant, or a pharmaceutically acceptable salt thereof, and at least one further pharmaceutical active ingredient, for use according to embodiment 14c or 15c, wherein mavoglurant, or a pharmaceutically acceptable salt thereof, is administered in the morning and in the evening separated by a 12 hour interval.
17c. A pharmaceutical combination comprising mavoglurant, or a pharmaceutically acceptable salt thereof, and at least one further pharmaceutical active ingredient, for use according to any of the preceding embodiments, wherein opioid use disorder is associated with binge drinking.

### Embodiments (d)

1d. Use of mavoglurant, or a pharmaceutically acceptable salt thereof, for the manufacture of a medicament for the reduction of opioid use by an opioid use disorder patient.
2d. Use of mavoglurant, or a pharmaceutically acceptable salt thereof, for the manufacture of a medicament for preventing relapse into opioid use by an opioid use disorder patient.
3d. Use of mavoglurant, or a pharmaceutically acceptable salt thereof, for the manufacture of a medicament for the promotion of opioid abstinence by an opioid use disorder patient.
4d. Use of mavoglurant, or a pharmaceutically acceptable salt thereof, for the manufacture of a medicament for the treatment of the symptoms of depression or anxiety associated with opioid use disorder.
5d. Use of mavoglurant, or a pharmaceutically acceptable salt thereof, according to any one of embodiments 1d to 4d, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
6d. Use of mavoglurant, or a pharmaceutically acceptable salt thereof, according to any one of embodiments 1d to 5d, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
7d. Use of mavoglurant, or a pharmaceutically acceptable salt thereof, according to any one of embodiments 1d to 6d, wherein the use is combined with standardized psychological treatment, for example, at individual or group level.
8d. Use of mavoglurant, or a pharmaceutically acceptable salt thereof, according to any one of embodiments 1d to 7d, wherein the use is combined with psychosocial or behavioral therapy or combination thereof, in particular contingency management based therapy.
9d. Use of mavoglurant, or a pharmaceutically acceptable salt thereof, according to embodiment 8d, wherein the psychosocial or the behavioral therapy is computer-assisted.
10d. Use of mavoglurant, or a pharmaceutically acceptable salt thereof, according to any one of embodiments 1d to 9d, wherein the use is concomitant with methadone or buprenorphine treatment.
11d. Use of mavoglurant, or a pharmaceutically acceptable salt thereof, according to any one of embodiments 1d to 10d, wherein mavoglurant, or a pharmaceutically acceptable salt thereof, is administered in combination with a further active agent.
12d. Use of mavoglurant, or a pharmaceutically acceptable salt thereof, according to embodiment 11d, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.
13d. Use of mavoglurant, or a pharmaceutically acceptable salt thereof, according to any one of embodiments 1d to 12d, wherein the patient has a genetic variation associated with a substance use disorder, such as a genetic variation associated with opioid use disorder.
14d. Use of mavoglurant, or a pharmaceutically acceptable salt thereof, according to any one of embodiments 1d to 13d, wherein mavoglurant, or a pharmaceutically acceptable salt thereof, is administered in an immediate-release form or a modified-release form.
15d. Use of mavoglurant, or a pharmaceutically acceptable salt thereof, according to any one of embodiments 1d to 14d, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
16d. Use of mavoglurant, or a pharmaceutically acceptable salt thereof, according to embodiment 15d, wherein mavoglurant, or a pharmaceutically acceptable salt thereof, is administered with food.
17d. Use of mavoglurant, or a pharmaceutically acceptable salt thereof, according to embodiment 15d or 16d, wherein mavoglurant, or a pharmaceutically acceptable salt thereof, is administered in the morning and in the evening separated by a 12 hour interval.
18d. Use of mavoglurant, or a pharmaceutically acceptable salt thereof, according to any of the preceding embodiments, wherein opioid use disorder is associated with binge drinking.

### Embodiments (e)

1e. Use of a pharmaceutical composition comprising mavoglurant, or a pharmaceutically acceptable salt thereof, and at least one pharmaceutically acceptable excipient, for the manufacture of a medicament for the reduction of opioid use by an opioid use disorder patient.
2e. Use of a pharmaceutical composition comprising mavoglurant, or a pharmaceutically acceptable salt thereof, and at least one pharmaceutically acceptable excipient, for the manufacture of a medicament for preventing relapse into opioid use by an opioid use disorder patient.
3e. Use of a pharmaceutical composition comprising mavoglurant, or a pharmaceutically acceptable salt thereof, and at least one pharmaceutically acceptable excipient, for the manufacture of a medicament for the promotion of opioid abstinence by an opioid use disorder patient.
4e. Use of a pharmaceutical composition comprising mavoglurant, or a pharmaceutically acceptable salt thereof, and at least one pharmaceutically acceptable excipient, for the manufacture of a medicament for the treatment of the symptoms of depression or anxiety associated with opioid use disorder.
5e. Use of a pharmaceutical composition comprising mavoglurant, or a pharmaceutically acceptable salt thereof, and at least one pharmaceutically acceptable excipient, according to any one of embodiments 1e to 4e, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
6e. Use of a pharmaceutical composition comprising mavoglurant, or a pharmaceutically acceptable salt thereof, and at least one pharmaceutically acceptable excipient, according to any one of embodiments 1e to 5e, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
7e. Use of a pharmaceutical composition comprising mavoglurant, or a pharmaceutically acceptable salt thereof, and at least one pharmaceutically acceptable excipient, according to any one of embodiments 1e to 6e, wherein the use is combined with standardized psychological treatment, for example, at individual or group level.
8e. Use of a pharmaceutical composition comprising mavoglurant, or a pharmaceutically acceptable salt thereof, and at least one pharmaceutically acceptable excipient, according to any one of embodiments 1e to 7e, wherein the use is combined with psychosocial or behavioral therapy or combination thereof, in particular contingency management based therapy.
9e. Use of a pharmaceutical composition comprising mavoglurant, or a pharmaceutically acceptable salt thereof, and at least one pharmaceutically acceptable excipient, according to embodiment 8e, wherein the psychosocial or the behavioral therapy is computer-assisted.
10e. Use of a pharmaceutical composition comprising mavoglurant, or a pharmaceutically acceptable salt thereof, and at least one pharmaceutically acceptable excipient, according to any one of embodiments 1e to 9e, wherein the use is concomitant with methadone or buprenorphine treatment.
11e. Use of a pharmaceutical composition comprising mavoglurant, or a pharmaceutically acceptable salt thereof, and at least one pharmaceutically acceptable excipient, according to any one of embodiments 1e to 10e, wherein mavoglurant, or a pharmaceutically acceptable salt thereof, is administered in combination with a further active agent.
12e. Use of a pharmaceutical composition comprising mavoglurant, or a pharmaceutically acceptable salt thereof, and at least one pharmaceutically acceptable excipient, according to embodiment 11e, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.
13e. Use of a pharmaceutical composition comprising mavoglurant, or a pharmaceutically acceptable salt thereof, and at least one pharmaceutically acceptable excipient, according to any one of embodiments 1e to 12e, wherein the patient has a genetic variation associated with a substance use disorder, such as a genetic variation associated with opioid use disorder.
14e. Use of a pharmaceutical composition comprising mavoglurant, or a pharmaceutically acceptable salt thereof, and at least one pharmaceutically acceptable excipient, according to any one of embodiments 1e to 13e, wherein mavoglurant, or a pharmaceutically acceptable salt thereof, is administered in an immediate-release form or a modified-release form.
15e. Use of a pharmaceutical composition comprising mavoglurant, or a pharmaceutically acceptable salt thereof, and at least one pharmaceutically acceptable excipient, according to any one of embodiments 1e to 14e, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
16e. Use of a pharmaceutical composition comprising mavoglurant, or a pharmaceutically acceptable salt thereof, and at least one pharmaceutically acceptable excipient, according to embodiment 15e, wherein mavoglurant, or a pharmaceutically acceptable salt thereof, is administered with food.
17e. Use of a pharmaceutical composition comprising mavoglurant, or a pharmaceutically acceptable salt thereof, and at least one pharmaceutically acceptable excipient, according to embodiment 15e or 16e, wherein mavoglurant, or a pharmaceutically acceptable salt thereof, is administered in the morning and in the evening separated by a 12 hour interval.
18e. Use of a pharmaceutical composition comprising mavoglurant, or a pharmaceutically acceptable salt thereof, and at least one pharmaceutically acceptable excipient, according to any of the preceding embodiments, wherein opioid use disorder is associated with binge drinking.

### Embodiments (f)

1f. Use of a pharmaceutical combination comprising mavoglurant, or a pharmaceutically acceptable salt thereof, and at least one further pharmaceutical active ingredient, for the manufacture of a medicament for the reduction of opioid use by an opioid use disorder patient.
2f. Use of a pharmaceutical combination comprising mavoglurant, or a pharmaceutically acceptable salt thereof, and at least one further pharmaceutical active ingredient, for the manufacture of a medicament for preventing relapse into opioid use by an opioid use disorder patient.
3f. Use of a pharmaceutical combination comprising mavoglurant, or a pharmaceutically acceptable salt thereof, and at least one further pharmaceutical active ingredient, for the manufacture of a medicament for the promotion of opioid abstinence by an opioid use disorder patient.
4f. Use of a pharmaceutical combination comprising mavoglurant, or a pharmaceutically acceptable salt thereof, and at least one further pharmaceutical active ingredient, for the manufacture of a medicament for the treatment of the symptoms of depression or anxiety associated with opioid use disorder.
5f. Use of a pharmaceutical combination comprising mavoglurant, or a pharmaceutically acceptable salt thereof, and at least one further pharmaceutical active ingredient, according to any one of embodiments 1f to 4f, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
6f. Use of a pharmaceutical combination comprising mavoglurant, or a pharmaceutically acceptable salt thereof, and at least one further pharmaceutical active ingredient, according to any one of embodiments 1f to 5f, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
7f. Use of a pharmaceutical combination comprising mavoglurant, or a pharmaceutically acceptable salt thereof, and at least one further pharmaceutical active ingredient, according to any one of embodiments 1f to 6f, wherein the use is combined with standardized psychological treatment, for example, at individual or group level.
8f. Use of a pharmaceutical combination comprising mavoglurant, or a pharmaceutically acceptable salt thereof, and at least one further pharmaceutical active ingredient, according to any one of embodiments 1f to 7f, wherein the use is combined with psychosocial or behavioral therapy or combination thereof, in particular contingency management based therapy.
9f. Use of a pharmaceutical combination comprising mavoglurant, or a pharmaceutically acceptable salt thereof, and at least one further pharmaceutical active ingredient, according embodiment 8f, wherein the psychosocial or the behavioral therapy is computer-assisted.
10f. Use of a pharmaceutical combination comprising mavoglurant, or a pharmaceutically acceptable salt thereof, and at least one further pharmaceutical active ingredient, according to any one of embodiments 1f to 9f, wherein the use is concomitant with methadone or buprenorphine treatment.
11f. Use of a pharmaceutical combination comprising mavoglurant, or a pharmaceutically acceptable salt thereof, and at least one further pharmaceutical active ingredient, according to any one of embodiments 1f to 10f, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.
12f. Use of a pharmaceutical combination comprising mavoglurant, or a pharmaceutically acceptable salt thereof, and at least one further pharmaceutical active ingredient, according to any one of embodiments 1f to 11f, wherein the patient has a genetic variation associated with a substance use disorder, such as a genetic variation associated with opioid use disorder.
13f. Use of a pharmaceutical combination comprising mavoglurant, or a pharmaceutically acceptable salt thereof, and at least one further pharmaceutical active ingredient, according to any one of embodiments 1f to 12f, wherein mavoglurant, or a pharmaceutically acceptable salt thereof, is administered in an immediate-release form or a modified-release form.
14f. Use of a pharmaceutical combination comprising mavoglurant, or a pharmaceutically acceptable salt thereof, and at least one further pharmaceutical active ingredient, according to any one of embodiments 1c to 13c, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
15f. Use of a pharmaceutical combination comprising mavoglurant, or a pharmaceutically acceptable salt thereof, and at least one further pharmaceutical active ingredient, according to embodiment 14f, wherein mavoglurant, or a pharmaceutically acceptable salt thereof, is administered with food.
16f. Use of a pharmaceutical combination comprising mavoglurant, or a pharmaceutically acceptable salt thereof, and at least one further pharmaceutical active ingredient, according to embodiment 14f or 15f, wherein mavoglurant, or a pharmaceutically acceptable salt thereof, is administered in the morning and in the evening separated by a 12 hour interval.
17f. Use of a pharmaceutical combination comprising mavoglurant, or a pharmaceutically acceptable salt thereof, and at least one further pharmaceutical active ingredient, according to any of the preceding embodiments, wherein opioid use disorder is associated with binge drinking.

### Embodiments (g)

1g. A method for the reduction of opioid use by an opioid use disorder patient, in need thereof, comprising administering to said opioid use disorder patient an effective amount of mavoglurant, or a pharmaceutically acceptable salt thereof.
2g. A method for preventing relapse into opioid use by an opioid use disorder patient, in need thereof, comprising administering to said opioid use disorder patient an effective amount of mavoglurant, or a pharmaceutically acceptable salt thereof.
3g. A method for the promotion of opioid abstinence by an opioid use disorder patient, in need thereof, comprising administering to said opioid use disorder patient an effective amount of mavoglurant, or a pharmaceutically acceptable salt thereof.
4g. A method for treating the symptoms of depression or anxiety associated with opioid use disorder by an opioid use disorder patient, in need thereof, comprising administering to said opioid use disorder patient an effective amount of mavoglurant, or a pharmaceutically acceptable salt thereof.
5g. A method according to any one of embodiments 1 g to 4g, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
6g. A method according to any one of embodiments 1 g to 5g, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
7g. A method according to any one of embodiments 1g to 6g, wherein administration of mavoglurant, or a pharmaceutically acceptable salt thereof is combined with standardized psychological treatment, for example, at individual or group level.
8g. A method according to any one of embodiments 1g to 7g, wherein administration of mavoglurant, or a pharmaceutically acceptable salt thereof is combined is combined with psychosocial or behavioral therapy or combination thereof, in particular contingency management based therapy.
9g. A method according to embodiment 8g, wherein the psychosocial or the behavioral therapy is computer-assisted.
10g. A method according to any one of embodiments 1g to 9g, wherein administration of mavoglurant, or a pharmaceutically acceptable salt thereof, is concomitant with methadone or buprenorphine treatment.
11g. A method according to any one of embodiments 1g to 10g, wherein mavoglurant, or a pharmaceutically acceptable salt thereof, is administered in combination with a further active agent.
12g. A method according to embodiment 11g, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.
13g. A method according to any one of embodiments 1g to 12g, wherein the patient has a genetic variation associated with a substance use disorder, such as a genetic variation associated with opioid use disorder.
14g. A method according to any one of embodiments 1g to 13g, wherein mavoglurant, or a pharmaceutically acceptable salt thereof, is administered in an immediate-release form or a modified-release form.
15g. A method according to any one of embodiments 1g to 14g, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
16g. A method according to embodiment 15g, wherein mavoglurant, or a pharmaceutically acceptable salt thereof, is administered with food.
17g. A method according to embodiment 15g or 16g, wherein mavoglurant, or a pharmaceutically acceptable salt thereof, is administered in the morning and in the evening separated by a 12 hour interval.
18g. A method according to any of the preceding embodiments, wherein opioid use disorder is associated with binge drinking.

### Embodiments (h)

1h. A method for the reduction of opioid use by an opioid use disorder patient, in need thereof, comprising administering to said opioid use disorder patient a pharmaceutical composition comprising an effective amount of mavoglurant, or a pharmaceutically acceptable salt thereof, and at least one pharmaceutically acceptable excipient.
2h. A method for preventing relapse into opioid use by an opioid use disorder patient, in need thereof, comprising administering to said opioid use disorder patient a pharmaceutical composition comprising an effective amount of mavoglurant, or a pharmaceutically acceptable salt thereof, and at least one pharmaceutically acceptable excipient.
3h. A method for the promotion of opioid abstinence by an opioid use disorder patient, in need thereof, comprising administering to said opioid use disorder patient a pharmaceutical composition comprising an effective amount of mavoglurant, or a pharmaceutically acceptable salt thereof, and at least one pharmaceutically acceptable excipient.
4h. A method for treating the symptoms of depression or anxiety associated with opioid use disorder by an opioid use disorder patient, in need thereof, comprising administering to said opioid use disorder patient a pharmaceutical composition comprising an effective amount of mavoglurant, or a pharmaceutically acceptable salt thereof, and at least one pharmaceutically acceptable excipient.
5h. A method according to any one of embodiments 1h to 4h, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
6h. A method according to any one of embodiments 1h to 5h, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
7h. A method according to any one of embodiments 1h to 6h, wherein administration of the pharmaceutical composition comprising an effective amount of mavoglurant, or a pharmaceutically acceptable salt thereof, and at least one pharmaceutically acceptable excipient, is combined with standardized psychological treatment, for example, at individual or group level.
8h. A method according to any one of embodiments 1h to 7h, wherein administration of the pharmaceutical composition comprising an effective amount of mavoglurant, or a pharmaceutically acceptable salt thereof, and at least one pharmaceutically acceptable excipient, is combined with psychosocial or behavioral therapy or combination thereof, in particular contingency management based therapy.
9h. A method according to embodiment 8h, wherein the psychosocial or the behavioral therapy is computer-assisted.
10h. A method according to any one of embodiments 1h to 9h, wherein administration of the pharmaceutical composition comprising an effective amount of mavoglurant, or a pharmaceutically acceptable salt thereof, and at least one pharmaceutically acceptable excipient, is concomitant with methadone or buprenorphine treatment.
11h. A method according to any one of embodiments 1h to 10h, wherein mavoglurant, or a pharmaceutically acceptable salt thereof, is administered in combination with a further active agent.
12h. A method according to embodiment 11h, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A}, receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.
13h. A method according to any one of embodiments 1h to 12h, wherein the patient has a genetic variation associated with a substance use disorder, such as a genetic variation associated with opioid use disorder.
14h. A method according to any one of embodiments 1h to 13h, wherein the pharmaceutical composition comprising mavoglurant, or a pharmaceutically acceptable salt thereof, and at least one pharmaceutically acceptable excipient, is an immediate-release form or a modified-release form.
15h. A method according to any one of embodiments 1h to 14h, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
16h. A method according to embodiment 15h, wherein the pharmaceutical composition comprising an effective amount of mavoglurant, or a pharmaceutically acceptable salt thereof, and at least one pharmaceutically acceptable excipient, is administered with food.
17h. A method according to embodiment 15h or 16h, wherein the pharmaceutical composition comprising an effective amount of mavoglurant, or a pharmaceutically acceptable salt thereof, and at least one pharmaceutically acceptable excipient, is administered in the morning and in the evening separated by a 12 hour interval.
18h. A method according to any of the preceding embodiments, wherein opioid use disorder is associated with binge drinking.

### Embodiments (j)

1j. A method for the reduction of opioid use by an opioid use disorder patient, in need thereof, comprising administering to said opioid use disorder patient a pharmaceutical combination comprising an effective amount of mavoglurant, or a pharmaceutically acceptable salt thereof, and at least one further pharmaceutical active ingredient.
2j. A method for preventing relapse into opioid use by an opioid use disorder patient, in need thereof, comprising administering to said opioid use disorder patient a pharmaceutical combination comprising an effective amount of mavoglurant, or a pharmaceutically acceptable salt thereof, and at least one at least one further pharmaceutical active ingredient.
3j. A method for the promotion of opioid abstinence by an opioid use disorder patient, in need thereof, comprising administering to said opioid use disorder patient a pharmaceutical combination comprising an effective amount of mavoglurant, or a pharmaceutically acceptable salt thereof, and at least one at least one further pharmaceutical active ingredient.
4j. A method for treating the symptoms of depression or anxiety associated with opioid use disorder by an opioid use disorder patient, in need thereof, comprising administering to said opioid use disorder patient a pharmaceutical combination comprising an effective amount of mavoglurant, or a pharmaceutically acceptable salt thereof, and at least one at least one further pharmaceutical active ingredient.
5j. A method according to any one of embodiments 1j to 4j, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
6j. A method according to any one of embodiments 1j to 5j, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
7j. A method according to any one of embodiments 1j to 6j, wherein administration of the pharmaceutical combination comprising an effective amount of mavoglurant, or a pharmaceutically acceptable salt thereof, and at least one further pharmaceutical active ingredient, is combined with standardized psychological treatment, for example, at individual or group level.
8j. A method according to any one of embodiments 1j to 7j, wherein administration of the pharmaceutical combination comprising an effective amount of mavoglurant, or a pharmaceutically acceptable salt thereof, and at least one further pharmaceutical active ingredient, is combined with psychosocial or behavioral therapy or combination thereof, in particular contingency management based therapy.
9j. A method according to embodiment 8j, wherein the psychosocial or the behavioral therapy is computer-assisted.
10j. A method according to any one of embodiments 1j to 9j, wherein administration of the pharmaceutical combination comprising an effective amount of mavoglurant, or a pharmaceutically acceptable salt thereof, and at least one further pharmaceutical active ingredient, is concomitant with methadone or buprenorphine treatment.
11j. A method according to embodiment 10j, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.
12j. A method according to any one of embodiments 1j to 11j, wherein the patient has a genetic variation associated with a substance use disorder, such as a genetic variation associated with opioid use disorder.
13j. A method according to any one of embodiments 1j to 12j, wherein the pharmaceutical combination comprising mavoglurant, or a pharmaceutically acceptable salt thereof, and at least one further pharmaceutical active ingredient, is an immediate-release form or a modified-release form.
14j. A method according to any one of embodiments 1j to 13j, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
15j. A method according to embodiment 14j, wherein the pharmaceutical combination comprising an effective amount of mavoglurant, or a pharmaceutically acceptable salt thereof, and at least one further pharmaceutical active ingredient, is administered with food.
16j. A method according to embodiment 14j or 15j, wherein the pharmaceutical combination comprising an effective amount of mavoglurant, or a pharmaceutically acceptable salt thereof, and at least one further pharmaceutical active ingredient, is administered in the morning and in the evening separated by a 12 hour interval.
17j. A method according to any of the preceding embodiments, wherein opioid use disorder is associated with binge drinking.

### Embodiments (k)

Embodiment 1k: Mavoglurant, or a pharmaceutically acceptable salt thereof, for use in the reduction of opioid use by an opioid use disorder patient.
Embodiment 2k: Mavoglurant, or a pharmaceutically acceptable salt thereof, for use in preventing relapse into opioid use by an opioid use disorder patient.
Embodiment 3k: Mavoglurant, or a pharmaceutically acceptable salt thereof, for use in the promotion of opioid abstinence by an opioid use disorder patient.
Embodiment 4k: Mavoglurant, or a pharmaceutically acceptable salt thereof, for use in the treatment of the symptoms of depression or anxiety associated with opioid use disorder.
Embodiment 5k: Mavoglurant, or a pharmaceutically acceptable salt thereof, for use according to any one of embodiments 1k to 4k, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
Embodiment 6k: Mavoglurant, or a pharmaceutically acceptable salt thereof, for use according to any one of embodiments 1k to 5k, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
Embodiment 7k: Mavoglurant, or a pharmaceutically acceptable salt thereof, for use according to any one of embodiments 1k to 6k, wherein the use is combined with standardized psychological treatment, for example, at individual or group level.
Embodiment 8k: Mavoglurant, or a pharmaceutically acceptable salt thereof, for use according to any one of embodiments 1k to 7k, wherein the use is combined with psychosocial or behavioral therapy or combination thereof, in particular contingency management based therapy.
Embodiment 9k: Mavoglurant, or a pharmaceutically acceptable salt thereof, for use according to embodiment 8k, wherein the psychosocial or the behavioral therapy is computer-assisted.
Embodiment 10k: Mavoglurant, or a pharmaceutically acceptable salt thereof, for use according to any one of embodiments 1k to 9k, wherein the use is concomitant with methadone or buprenorphine treatment.
Embodiment 11k: Mavoglurant, or a pharmaceutically acceptable salt thereof, for use according to any one of embodiments 1k to 10k, wherein mavoglurant, or a pharmaceutically acceptable salt thereof, is administered in combination with a further active agent.
Embodiment 12k: Mavoglurant, or a pharmaceutically acceptable salt thereof, for use according to any one of embodiments 1k to 11k, wherein the patient has a genetic variation associated with a substance use disorder, such as a genetic variation associated with opioid use disorder.
Embodiment 13k: Mavoglurant, or a pharmaceutically acceptable salt thereof, for use according to any one of embodiments 1k to 12k, wherein mavoglurant, or a pharmaceutically acceptable salt thereof, is administered in an immediate-release form or a modified-release form.
Embodiment 14k: Mavoglurant, or a pharmaceutically acceptable salt thereof, for use according to any one of embodiments 1k to 13k, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
Embodiment 15k: Mavoglurant, or a pharmaceutically acceptable salt thereof, for use according to any of the preceding embodiments, wherein opioid use disorder is associated with binge drinking.

### Embodiments (m)

1m. Mavoglurant, or a pharmaceutically acceptable salt thereof, for use in the treatment of opioid use disorder.
2m. Mavoglurant, or a pharmaceutically acceptable salt thereof, for use according to embodiment 1m, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
3m. Mavoglurant, or a pharmaceutically acceptable salt thereof, for use according to any one of embodiments 1m or 2m, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
4m. Mavoglurant, or a pharmaceutically acceptable salt thereof, for use according to any one of embodiments 1m to 3m, wherein the use is combined with standardized psychological treatment, for example, at individual or group level.
5m. Mavoglurant, or a pharmaceutically acceptable salt thereof, for use according to any one of embodiments 1m to 4m, wherein the use is combined with psychosocial or behavioral therapy or combination thereof, in particular contingency management based therapy.
6m. Mavoglurant, or a pharmaceutically acceptable salt thereof, for use according to embodiment 5m, wherein the psychosocial or the behavioral therapy is computer-assisted.
7m. Mavoglurant, or a pharmaceutically acceptable salt thereof, for use according to any one of embodiments 1m to 6m, wherein the use is concomitant with methadone or buprenorphine treatment.
8m. Mavoglurant, or a pharmaceutically acceptable salt thereof, for use according to any one of embodiments 1m to 7m, wherein mavoglurant, or a pharmaceutically acceptable salt thereof, is administered in combination with a further active agent.
9m. Mavoglurant, or a pharmaceutically acceptable salt thereof, for use according to embodiment 8m, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.
10m. Mavoglurant, or a pharmaceutically acceptable salt thereof, for use according to any one of embodiments 1m to 9m, wherein the patient has a genetic variation associated with a substance use disorder, such as a genetic variation associated with opioid use disorder.
11m. Mavoglurant, or a pharmaceutically acceptable salt thereof, for use according to any one of embodiments 1m to 10m, wherein mavoglurant, or a pharmaceutically acceptable salt thereof, is administered in an immediate-release form or a modified-release form.
12m. Mavoglurant, or a pharmaceutically acceptable salt thereof, for use according to any one of embodiments 1m to 11m, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
13m. Mavoglurant, or a pharmaceutically acceptable salt thereof, for use according to embodiment 12m, wherein mavoglurant, or a pharmaceutically acceptable salt thereof, is administered with food.
14m. Mavoglurant, or a pharmaceutically acceptable salt thereof, for use according to embodiment 12m or 13m, wherein mavoglurant, or a pharmaceutically acceptable salt thereof, is administered in the morning and in the evening separated by a 12 hour interval.
15m. Mavoglurant, or a pharmaceutically acceptable salt thereof, for use according to any of the preceding embodiments, wherein opioid use disorder is associated with binge drinking.

### Embodiments (n)

1n. Use of mavoglurant, or a pharmaceutically acceptable salt thereof, for the manufacture of a medicament for the treatment of opioid use disorder.
2n. Use of mavoglurant, or a pharmaceutically acceptable salt thereof, according to embodiment 1n, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
3n. Use of mavoglurant, or a pharmaceutically acceptable salt thereof, according to embodiments 1n or 2n, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
4n. Use of mavoglurant, or a pharmaceutically acceptable salt thereof, according to any one of embodiments 1n to 3n, wherein the use is combined with standardized psychological treatment, for example, at individual or group level.
5n. Use of mavoglurant, or a pharmaceutically acceptable salt thereof, according to any one of embodiments 1n to 4n, wherein the use is combined with psychosocial or behavioral therapy or combination thereof, in particular contingency management based therapy.
6n. Use of mavoglurant, or a pharmaceutically acceptable salt thereof, according to embodiment 5n, wherein the psychosocial or the behavioral therapy is computer-assisted.
7n. Use of mavoglurant, or a pharmaceutically acceptable salt thereof, according to any one of embodiments 1n to 9n, wherein the use is concomitant with methadone or buprenorphine treatment.
8n. Use of mavoglurant, or a pharmaceutically acceptable salt thereof, according to any one of embodiments 1n to 7n, wherein mavoglurant, or a pharmaceutically acceptable salt thereof, is administered in combination with a further active agent.
9n. Use of mavoglurant, or a pharmaceutically acceptable salt thereof, according to embodiment 8n, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.
10n. Use of mavoglurant, or a pharmaceutically acceptable salt thereof, according to any one of embodiments 1n to 9n, wherein the patient has a genetic variation associated with a substance use disorder, such as a genetic variation associated with opioid use disorder.
11n. Use of mavoglurant, or a pharmaceutically acceptable salt thereof, according to any one of embodiments 1n to 10n, wherein mavoglurant, or a pharmaceutically acceptable salt thereof, is administered in an immediate-release form or a modified-release form.
12n. Use of mavoglurant, or a pharmaceutically acceptable salt thereof, according to any one of embodiments 1n to 11n, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
13n. Use of mavoglurant, or a pharmaceutically acceptable salt thereof, according to embodiment 12n, wherein mavoglurant, or a pharmaceutically acceptable salt thereof, is administered with food.
14n. Use of mavoglurant, or a pharmaceutically acceptable salt thereof, according to embodiment 12n or 13n, wherein mavoglurant, or a pharmaceutically acceptable salt thereof, is administered in the morning and in the evening separated by a 12 hour interval.
15n. Use of mavoglurant, or a pharmaceutically acceptable salt thereof, according to any of the preceding embodiments, wherein opioid use disorder is associated with binge drinking.

### Embodiments (p)

1p. A method for treating opioid use disorder patient, in a patient in need thereof, comprising administering to said opioid use disorder patient an effective amount of mavoglurant, or a pharmaceutically acceptable salt thereof.
2p. A method according to embodiment 1p, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).
3p. A method according to embodiments 1p or 2p, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.
4p. A method according to any one of embodiments 1p to 3p, wherein administration of mavoglurant, or a pharmaceutically acceptable salt thereof is combined with standardized psychological treatment, for example, at individual or group level.
5p. A method according to any one of embodiments 1p to 4p, wherein administration of mavoglurant, or a pharmaceutically acceptable salt thereof is combined is combined with psychosocial or behavioral therapy or combination thereof, in particular contingency management based therapy.
6p. A method according to embodiment 5p, wherein the psychosocial or the behavioral therapy is computer-assisted.
7p. A method according to any one of embodiments 1p to 6p, wherein administration of mavoglurant, or a pharmaceutically acceptable salt thereof, is concomitant with methadone or buprenorphine treatment.
8p. A method according to any one of embodiments 1p to 7p, wherein mavoglurant, or a pharmaceutically acceptable salt thereof, is administered in combination with a further active agent.
9p. A method according to embodiment 8p, wherein the further active agent is selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.
10p. A method according to any one of embodiments 1p to 9p, wherein the patient has a genetic variation associated with a substance use disorder, such as a genetic variation associated with opioid use disorder.
11p. A method according to any one of embodiments 1p to 10p, wherein mavoglurant, or a pharmaceutically acceptable salt thereof, is administered in an immediate-release form or a modified-release form.
12p. A method according to any one of embodiments 1p to 11p, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
13p. A method according to embodiment 12p, wherein mavoglurant, or a pharmaceutically acceptable salt thereof, is administered with food.
14p. A method according to embodiment 12p or 13p, wherein mavoglurant, or a pharmaceutically acceptable salt thereof, is administered in the morning and in the evening separated by a 12 hour interval.
15p. A method according to any of the preceding embodiments, wherein opioid use disorder is associated with binge drinking.

### General Terms

The term “opioid use disorder” or “OUD”, as used herein, is defined with reference to DSM-5 criteria (i.e. according to the Diagnostic and Statistical Manual of Mental Disorders. 5th Edition, Washington, D.C.: American Psychiatric Association, 2013), the entire contents of which are incorporated herein by reference. As used herein, the term “opioid use disorder” is defined as a problematic pattern of opioid use leading to clinically significant impairment or distress, as manifested by at least two of the following, occurring within a 12-month period:
1) Opioids are often taken in larger amounts or over a longer period than was intended.
2) There is a persistent desire or unsuccessful efforts to cut down or control opioid use.
3) A great deal of time is spent in activities necessary to obtain the opioid, use the opioid, or recover from its effects.
4) Craving, or a strong desire or urge to use opioids,
5) Recurrent opioid use resulting in a failure to fulfill major role obligations at work, school, or home.
6) Continued opioid use despite having persistent or recurrent social or interpersonal problems caused or exacerbated by the effects of opioids.
7) Important social, occupational, or recreational activities are given up or reduced because of opioid use.
8) Recurrent opioid use in situations in which it is physically hazardous.
9) Continued opioid use despite knowledge of having a persistent or recurrent physical or psychological problem that is likely to have been caused or exacerbated by opioids.
10) Tolerance, as defined by either of the following; a) A need for markedly increased amounts of opioids to achieve intoxication or desired effect; b) A markedly diminished effect with continued use of the same amount of an opioid.
11) Withdrawal, as manifested by either of the following:
   a) The characteristic opioid withdrawal syndrome:
      i) cessation of (or reduction in) prolonged opioid use;
      ii) dysphoric mood and two (or more) of the following physiological changes, developing within a few hours to several days after the cessation of (or reduction in) opioid use: fatigue; vivid, unpleasant dreams; insomnia or hypersomnia; increased appetite; psychomotor retardation or agitation.
   b) Opioids are taken to relieve or avoid withdrawal symptoms.
      “Opioid use disorder” may be separated into the following three categories: mild (i.e. presence of 2 or 3 symptoms, defined with reference to DSM-5 criteria), moderate (i.e. presence of 4 or 5 symptoms, defined with reference to DSM-5 criteria) and severe (i.e. presence of 6 or more symptoms, defined with reference to DSM-5 criteria). In one embodiment “opioid use disorder”, as used herein, refers to “mild opioid use disorder”, “moderate opioid use disorder” and “severe opioid use disorder”. In a further embodiment, “opioid use disorder”, as used herein, refers to “mild opioid use disorder”, “moderate opioid use disorder” or “severe opioid use disorder”.

The term “opioid use disorder patient” refers to a patient diagnosed with OUD, as defined herein. In one embodiment, the term “opioid use disorder patient” refers to a patient diagnosed with OUD who is in abstinence from opioid, for example, for at least 1 day, such as 3 days or more. The term “opioid use disorder patient in abstinence from opioid” refers to a patient diagnosed with OUD in abstinence from opioid for a period, for example, for at least 1 day. The term “opioid use disorder associated with binge drinking” refers to a patient that is diagnosed with opioid use disorder and is an abuser of alcohol (i.e. a heavy drinker). As explained at http://drugabuse.com/library/alcohol-abuse/, abusers of alcohol may not drink on a consistent basis, for example, they may only drink once a week, but, when drinking, they may drink heavily, which will cause problems, such as suffering from alcohol intoxication. For the sake of clarity, herein, an abuser of alcohol is not an alcohol use disorder patient (i.e. does not meet criteria for alcohol use disorder as defined with reference to DSM-5 criteria). The term “heavy drinker” refers to someone with a heavy alcohol use pattern. According to the National Institute on Alcohol Abuse and Alcoholism (NIAAA), the Substance Abuse and Mental Health Services Administration (SAMHSA) defines “heavy alcohol use” as binge drinking on 5 or more days in the past month. NIAAA defines binge drinking as a pattern of drinking that brings blood alcohol concentration (BAC) levels to 0.08 g/dL. This typically occurs after 4 alcoholic drinks for women and 5 alcoholic drinks for men—in about 2 hours. The Substance Abuse and Mental Health Services Administration (SAMHSA), defines “binge drinking” as 5 or more alcoholic drinks for males or 4 or more alcoholic drinks for females on the same occasion (i.e., at the same time or within a couple of hours of each other) on at least 1 day in the past month. The term “alcohol”, as used herein, for example in relation to “drinks”, “alcoholic drinks” or “drinking”, refers to ethyl alcohol (i.e. ethanol). The term “drinking”, “drinks” or “alcoholic drinks”, as used herein, is understood in the context of “standard drinks”, such as spirits or blends that are intended for human consumption, wherein a “standard drink” equals 12 g ethanol.

The term “opioid” as used herein, refers to both natural (opiate) or synthetic (opioids) forms, as well as natural or synthetic mixed opioid agonists/antagonists, and include, without limitation, alphamethylfentanyl, alfentanil, buprenorphine, butorphanol, codeine, diacetylmorphine, dihydrocodeine, dihydroetorphine, dihydromorphine, ethylmorphine, etorphine, fentanyl, hydrocodone, hydromorphone, L-acetylmethadol, levorphanol, methadone, meperidine, morphine, nicomorphine, normethadone, noroxycodone, normorphine, norlevorphanol, oxycodone, oxymorphone, phenazocine, propoxyphene, remifentanil, tramadol, thebaine, tapentadol, levorphanol, sufentanil, pentazocine, carfentanyl, ohmfentanyl, nocaine, ketobemidone, allylprodine, prodine, dextropropoxyphene, dextromoramide, benzitramide, piritramide, dipipanone, loperamide, diphenoxylate, nalbuphine, levomethorpha, dezocine, lefetamine and tildine. In one embodiment, “opioid” is selected from the group consisting of morphine, hydrocodone, oxycodone and codeine; in particular the opioid is selected from the group consisting of hydrocodone and oxycodone. In one embodiment, the opioid is hydrocodone. In another embodiment, the opioid is oxycodone.

The term “opioid craving” as used herein refers to a conscious desire or urge to consume an opioid.

The term “opioid use”, as used herein, refers to opioid consumption.

The term “reducing opioid use” or “reduction of opioid use”, as used herein, refers to reducing the amount or frequency of opioid use, for example as assessed by urinalysis (e.g. by measuring metabolites of opioids in urine) or as assessed by using self-reported opioid use with standardized tools like the Timeline Follow-Back self-report (Sobell L C, Sobell M B, 1996, Timeline FollowBack user's guide: A calendar method for assessing alcohol and drug use. Addiction Research Foundation, Toronto, Ontario, Canada; J. Anal. Toxicolo., 2002, 26: 393-400).

The term “opioid abstinence” or “in abstinence from opioid”, as used herein, refers to not taking opioids. The term “promoting opioid abstinence” or “promotion of opioid abstinence”, as used herein, refers to help maintaining abstinence from opioid use, in particular after at least 1 day of not taking opioids, for example maintaining abstinence from opioid use for a period of, for example, at least 1 week, 2 weeks, 3 weeks, 1 month, 3 months, 6 months or more, in particular at least 1 week or more, such as 2 weeks.

The term “relapse into opioid use” or “relapse into opioid consumption”, as used herein, refers to opioid intake (i.e. taking an opioid) following a period of opioid abstinence, for example following a period of opioid abstinence of at least 1 day or more, such as 3 days, 1 week, 2 weeks, 3 weeks, 1 month, 3 months, 6 months or more.

The term “preventing relapse into opioid use” or “preventing relapse into opioid consumption”, as used herein, refers to the prevention of opioid intake by a OUD patient after the patient has stopped the intake of opioids, in particular after 1 day or more of not taking opioids. In some embodiments, the term encompasses the permanent stoppage of opioid intake. In other embodiments, the term encompasses a delay in the resumption of opioid intake as compared to the time to resumption by a subject that is not administered a compound of the invention. The delay in resumption can be, e.g., days (e.g., 2, 3, 4, 5, 6, 7 days), weeks (e.g., 1, 2, 3 weeks), months (e.g., 1, 2, 3, 4, 5, 6 months), or longer.

The term “psychosocial or behavioral therapy”, as used herein, refers to, but not limited to, cognitive behavioral therapy (e.g. as described in Arch. Gen. Psychiatry 1999; 56:493-502), interpersonal therapy (e.g. as described in Psychol Addict Behav 2009; 23(1): 168-174), contingency management based therapy (e.g. as described in Psychol Addict Behav 2009; 23(1): 168-174; in J. Consul. Clin. Psychol. 2005; 73(2): 354-59; or in Case Reports in Psychiatry, Vol. 2012, Article ID 731638), community reinforcement approach based therapy (e.g. as described in Drug Alcohol Depend 2004; 74:1-13), motivational interviewing based therapy (e.g. as described in J. Consul. Clin. Psychol. 2001; 69(5): 858-62), motivational enhancement based therapy (e.g. as described in Drug Alcohol Depend 2007, 91:97-101) or meditation based therapy, such as transcendental meditation based therapy (e.g. as described in Addiction 2004; 99(7):862-874 or J. Consul. Clin. Psychol. 2000; 68(3): 515-52); in particular contingency management based therapy.

The term “standardized psychological treatment” or “standardized psychological support”, as used herein, refers to standard counselling sessions, for example once a week, in particular counselling focused on opioid consumption.

The term “computer-assisted” in the expression “the psychosocial or the behavioral therapy is computer-assisted”, as used herein, refers to psychosocial or behavioral therapy comprising the use of electronic tools such as online tools, smartphones, wireless devices or health Apps. In one embodiment, the term “computer-assisted” in the expression “the psychosocial or the behavioral therapy is computer-assisted”, as used herein, is to be understood as “computer-implemented” (i.e. the psychosocial or the behavioral therapy is computer-implemented).

The term “administered with food” refers to, for example, any food product, solid or liquid, with caloric content. The dosage of the mavoglurant, or pharmaceutically acceptable salt thereof, may be administered to a subject, for example, between thirty minutes prior to eating food, to, for example, one hour after consumption. In particular, administration of mavoglurant, or pharmaceutically acceptable salt thereof, occurs immediately after consuming food up to about thirty minutes after consumption.

The term “genetic variation” refers to a change in a gene sequence relative to a reference sequence (e.g., a commonly-found and/or wild-type sequence). Genetic variation may be recombination events or mutations such as substitution/deletion/insertion events like point and splice site mutations. In one embodiment, the genetic variation is a genetic variation in mGluR5.

The term “treat” “treating” “treatment” or “therapy”, as used herein, means obtaining beneficial or desired results, for example, clinical results. Beneficial or desired results can include, but are not limited to, alleviation of one or more symptoms of opioid use disorder patients, as defined herein, such as anxiety symptoms or depression symptoms associated with opioid use disorder, in particular by an opioid use disorder patient in abstinence from opioid, as herein defined. One aspect of the treatment is, for example, that said treatment should have a minimal adverse effect on the patient, e.g. the agent used should have a high level of safety, for example without producing the side effects of previously known treatment regimens. The term “alleviation”, for example in reference to a symptom of a condition, as used herein, refers to reducing at least one of the frequency and amplitude of a symptom of a condition in a patient.

The term “concomitant”, as used herein, for example in relation to “concomitant with methadone or buprenorphine treatment”, refers to both simultaneous and sequential administration.

As used herein, the term “subject” refers to a mammalian organism, preferably a human being (male or female).

As used herein, the term “patient” refers to a subject who is diseased and would benefit from the treatment.

As used herein, a subject is “in need of” a treatment if such subject (patient) would benefit biologically, medically or in quality of life from such treatment.

The term “pharmaceutical composition” is defined herein to refer to a mixture or solution containing at least one active ingredient or therapeutic agent to be administered to a subject, in order to treat a particular condition (i.e. disease, disorder or condition or at least one of the clinical symptoms thereof) affecting the subject.

As used herein, the term “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 22^{nd} Ed. Mack Printing Company, 2013, pp. 1049-1070). Except insofar as any conventional carrier is incompatible with the active ingredient, its use in the therapeutic or pharmaceutical compositions is contemplated.

The terms “drug”, “active substance”, “active ingredient”, “pharmaceutically active ingredient”, “active agent” or “therapeutic agent” are to be understood as meaning a compound in free form or in the form of a pharmaceutically acceptable salt, in particular compounds of the type specified herein. In particular, reference to mavoglurant, or a pharmaceutically acceptable salt thereof, in combination with a further active agent, as used herein (e.g. in any of embodiments (a) to (j), herein above, or in any of the claims, herein below), refers to mavoglurant in combination with at least one further active agent selected from a GABA agent (e.g. topiramate, tiagabine, baclofen or vigabatrin), cannabinoid CB1 receptor antagonist, a dual dopamine-serotonin releaser (e.g. PAL-287), a noradregenic agent (e.g. doxazosin, disulfiram or nepicastat), a 5-HT_{1A} receptor partial agonist (e.g. buspirone), a MU opioid agonist (e.g. buprenorophine), a NET inhibitor (e.g. desipramine) and a NMDA receptor antagonist (e.g. amantadine); or pharmaceutically acceptable salts thereof.

The term “immediate release form” refers to a pharmaceutical composition designed to release the active substance immediately upon in vivo administration.

The term “modified release form” refers to a pharmaceutical composition which releases the active substance not immediately, but offers a sustained, retard, continuous, gradual, prolonged or pulsatile release and therefore alters drug plasma levels distinctively versus an immediate release form. The term “modified release form” encompasses forms that are described as controlled-release form, sustained-release form, extended-release form; and long-acting form; in particular a sustained-release form.

The term “combination” or “pharmaceutical combination” refers to either a fixed combination in one unit dosage form (e.g., capsule, tablet, caplets or particulates), non-fixed combination, or a kit of parts for the combined administration where a compound of the present invention and one or more combination partner (e.g. another drug as specified herein, also referred to as further “pharmaceutical active ingredient”, “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. the compound of the present invention and one or more combination partners, are both administered to a patient simultaneously in the form of a single entity or dosage. The term “non-fixed combination” means that the active ingredients, e.g. a compound of the present invention and one or more combination partners, are both administered to a patient as separate entities either simultaneously or sequentially with no specific time limits, wherein such administration provides therapeutically effective levels of the two compounds in the body of the patient.

The compound of the present invention may be administered separately, by the same or different route of administration, or together in the same pharmaceutical composition as the other agents. In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

As used herein, the term “a,” “an,” “the” and similar terms used in the context of the present invention (especially in the context of the claims) are to be construed to cover both the singular and plural unless otherwise indicated herein or clearly contradicted by the context.

The use of any and all examples, or exemplary language (e.g. “such as”) provided herein is intended merely to better illuminate the invention and does not pose a limitation on the scope of the invention otherwise claimed.

As used herein, the compound of the invention, alternatively named Compound (I), as used herein above and below, is the mGluR5 antagonist (−)-(3aR,4S,7aR)-4-Hydroxy-4-m-tolylethynyl-octahydro-indole-1-carboxylic acid methyl ester, also named (−)-(3aR,4S,7aR)-4-Hydroxy-4-[2-(3-methylphenyl)ethynyl]perhydroindole-1-carboxylic acid methyl ester, also known as mavoglurant, of formula:

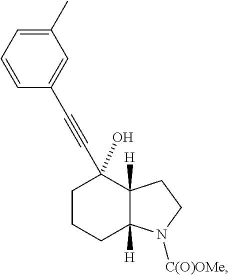

which can be e.g. prepared as described in WO2003/047581, e.g., in Example 1, or as described in WO2010/018154. WO2003/047581, which is incorporated herein by reference, also describes its in-vitro biological data, as per page 7. As used herein, “mavoglurant” refers to the free form, and any reference to “a pharmaceutically acceptable salt thereof” refers to a pharmaceutically acceptable acid addition salt thereof. As used herein, the term “mavoglurant, or a salt thereof, such as a pharmaceutically acceptable salt thereof”, as used in the context of the present invention (especially in the context of the any of the embodiments, above or below, and the claims) is thus to be construed to cover both the free form and a pharmaceutically acceptable salt thereof, unless otherwise indicated herein.

In one embodiment, Compound (I) is also intended to represent isotopically labeled forms. Isotopically labeled compounds have structures depicted by the formula above except that one or more atoms are replaced by an atom having a selected atomic mass or mass number. Isotopes that can be incorporated into the compound of the invention include, for example, isotopes of hydrogen, namely the compound of formula:

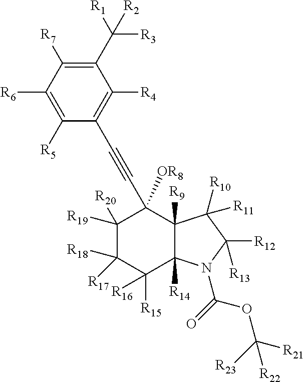

wherein each R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂ and R₂₃ is independently selected from H or deuterium; provided that there is at least one deuterium present in the compound. In other embodiments there are multiple deuterium atoms present in the compound.

Further, incorporation of certain isotopes, particularly deuterium (i.e., 2H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of the compound of the invention. The concentration of deuterium, may be defined by the isotopic enrichment factor. The term “isotopic enrichment factor” as used herein means the ratio between the isotopic abundance and the natural abundance of a specified isotope. If a substituent in the compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into the compound of the invention include isotopes of hydrogen, other than deuterium, carbon, nitrogen, oxygen, and fluorine such as ³H, ¹¹C, ¹³C, ¹⁴C, ¹⁵N, ¹⁸F respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. The isotopically-labeled compounds can generally be prepared by conventional techniques known to those skilled in the art or by processes analogous to those described preparation of the compound of the invention by using an appropriate isotopically-labeled reagents in place of the non-labeled reagent previously employed.

As used herein, the terms “free form” or “free forms” refers to the compound in non-salt form, such as the base free form or the acid free form of a respective compound, e.g. the compounds specified herein (e.g. mavoglurant or further pharmaceutical active ingredient, for example, as defined herein).

As used herein, the terms “salt”, “salts” or “salt form” refers to an acid addition or base addition salt of a respective compound, e.g. the compounds specified herein (e.g. mavoglurant or further pharmaceutical active ingredient, for example, as defined herein). “Salts” include in particular “pharmaceutically acceptable salts”. The term “pharmaceutically acceptable salts” refers to salts that retain the biological effectiveness and properties of the compounds and, which typically are not biologically or otherwise undesirable. The compounds, as specified herein (e.g. mavoglurant or further pharmaceutical active ingredient, for example, as defined herein), may be capable of forming acid and/or base salts by virtue of the presence of amino and/or carboxyl groups or groups similar thereto. The compound of the invention is capable of forming acid addition salts, thus, as used herein, the term pharmaceutically acceptable salt of mavoglurant means a pharmaceutically acceptable acid addition salt of mavoglurant.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca, Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 22^{nd} edition, Mack Publishing Company (2013); and in “Handbook of Pharmaceutical Salts: Properties, Selection, and Use” by Stahl and Wermuth (Wiley-VCH, Weinheim, 2011, 2^{nd} edition).

The compounds specified herein (e.g. mavoglurant or the further pharmaceutical active ingredient, for example, as defined herein) can be administered by conventional route, in particular orally, such as in the form of tablets, capsules, caplets or particulates, which can be manufactured according to pharmaceutical techniques as known in the art (for example in “Remington Essentials of Pharmaceutics, 2013, 1^{st} Edition, edited by Linda Felton, published by Pharmaceutical Press 2012, ISBN 978 0 85711 105 0; in particular Chapter 30), wherein pharmaceutical excipients are, for example, as described in “Handbook of Pharmaceutical Excipients, 2012, 7^{th} Edition, edited by Raymond C. Rowe, Paul J. Sheskey, Walter G. Cook and Marian E. Fenton, ISBN 978 0 85711 027 5”. In particular, WO2014/199316 describes formulations comprising mavoglurant, in particular modified release formulations thereof, and is incorporated herein by reference, more particularly the Examples, the preferred embodiments and claims therein.

The pharmaceutical composition or combination of the present invention can be in a unit dosage form (e.g. tablet, capsule, caplet or particulate) comprising an amount ranging of from 1 mg to 300 mg, in particular of from 50 mg to 200 mg, such as 50 mg to 100 mg, more particularly 200 mg, of mavoglurant (referring to an amount of the free form of mavoglurant, and if a salt thereof is used the amount will be adapted accordingly; in particular mavoglurant is in the free form). For the above-mentioned uses/treatment methods the appropriate dosage may vary depending upon a variety of factors, such as, for example, the age, weight, sex, the route of administration or salt employed. In patients with, for example, of from 50-70 kg body weight, an indicated daily dosage is, for example, 200 mg/b.i.d (referring to an amount of the free form of mavoglurant, and if a salt thereof is used the amount will be adapted accordingly).

### Abbreviations

BE=Benzoylecgonine
EtG=Ethyl Glucuronide
CM=Contingency Management
DSM 5=Diagnostic and Statistical Manual of Mental Disorders, 5th Ed.
OUD=Opioid Use Disorder
PK=Pharmacokinetic
TLFB=Timeline Follow-Back
mg=milligram
bid=b.i.d=twice (two times) a day
mmHg=millimeter of mercury
msec=millisecond
HIV=human immunodeficiency virus
ELISA=enzyme-linked immunosorbent assay
ECG=electrocardiogram
QT=time between the start of the Q wave and the end of the T wave
T wave=positive deflection after each QRS complex
ST wave=time between the start of the S wave and the end of the T wave
QRS=time between the start of the Q wave and the end of the T wave
QTcF=Fridericia QT correction formula
SoA=standard of care
SSRI=Selective serotonin reuptake inhibitors
C-SSRS=Columbia Suicide Severity Rating Scale
hCG=human chorionic gonadotropin
AST=aspartate aminotransferase
ALT=alanine aminotransferase
ULN=upper limit of normal
GGT=gamma-glutamyl transpeptidase
AV block=Atrioventricular block
UDS=urinary drug screening
MDMA=3,4-methylenedioxy-methamphetamine
MDEA=3,4-methylenedioxy-N-ethylamphetamine
MDA=3,4-methylenedioxy-amphetamine
° C.=degree Celsius
cm=centimeter
i.p.=intraperitoneal
p.o.=by mouth, orally
PEG=assessment of pain intensity (P), enjoyment of life (E), general activity (G)
NRS=Numeric Rating Scale
TLFB-Opioid=Timeline Follow Back Opioid Self-Reported Diary
COWS=Clinical Opiate Withdrawal Scale
OCS=Opioid Craving Scale
NOSE=Numerical Opioid Side Effect
QTcF=QT interval corrected by Fridericia's formula
QTcB=QT interval corrected by Bazett's formula
GGT=Gamma-glutamyl transferase
ULN=upper limit of normal
hCG=Human chorionic gonadotropin
FDA=Food and Drug Administration

The following Examples serve to illustrate the invention without limiting the scope thereof.

### EXAMPLES

### Example 1

The term “Compound (I)” (i.e. mavoglurant), as used in the context of these examples, refers to the free form. The term “Compound (III)” refers to 2-Methyl-6-(phenylethynyl)-pyridine (i.e. MPEP), which as used in the context of these examples, refers to the free form.

### Example 1a: Opiate Tolerance Test (Hot Plate) in the Mouse

The method, which detects tolerance to analgesic activity, follows that described by Fernandes et al (Naunyn-Schmiedeberg's Arch. Pharmacol., 297, 53-60, 1977).

Mice were placed onto a hot metal plate maintained at 54° C. surrounded by a Plexiglas cylinder (height: 13 cm; diameter: 19 cm) (Bioseb: model PE34). The latency to the first foot-lick was measured (maximum: 30 seconds). Morphine (32 mg/kg i.p.) or vehicle were administered twice daily for 6 days (at about 9:00 and 15:00), and then administered with morphine 8 mg/kg i.p. (or saline) 30 minutes before the test on Day 7 to assess morphine tolerance.

Compound (I) was evaluated at 3 doses (1, 3 and 10 mg/kg), administered in morphine tolerant mice p.o. acutely 60 minutes before the test on Day 7. The experiment included appropriate control groups to assess the potential effects of the test substance on morphine tolerance. Compound (III) was evaluated at 1 dose (10 mg/kg), administered under the same experimental conditions and was used as comparison substance.

10 mice were studied per group. The test was performed blind. Data were analysed by comparing treated groups with appropriate controls using unpaired Student's t tests.

### Results:

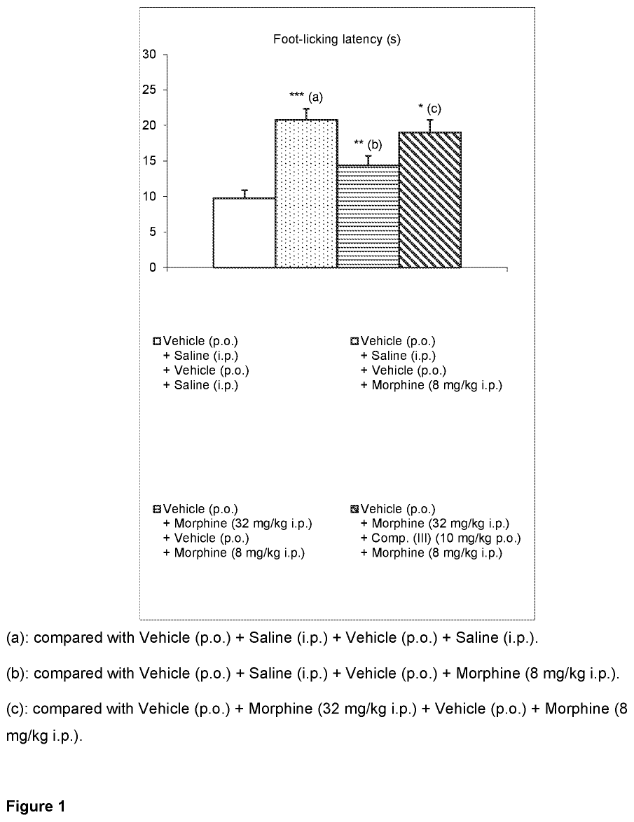

Compound (III) (10 mg/kg), administered p.o. 60 minutes before the test on Day 7 in animals receiving vehicle p.o. and morphine (32 mg/kg) i.p. twice daily from Day 1 to Day 6, significantly increased the foot-licking latency, as compared with animals receiving morphine from Day 1 to Day 6 (+32%, p<0.05) (FIG. 1).

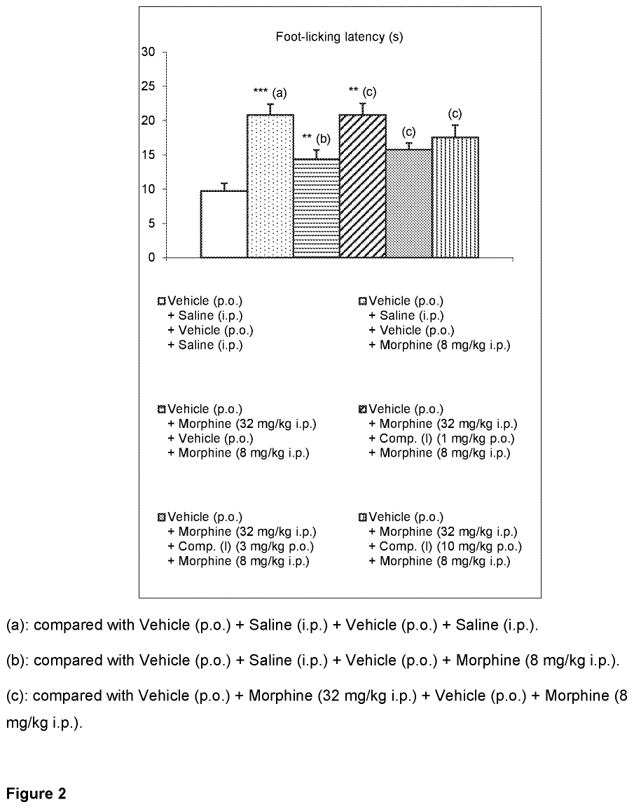

Compound (I) (1 mg/kg) administered p.o. 60 minutes before the test on Day 7 in animals receiving vehicle p.o. and morphine (32 mg/kg) i.p. twice daily from Day 1 to Day 6, significantly increased the foot-licking latency, as compared with animals receiving morphine from Day 1 to Day 6 (+44%, p<0.01). It had no significant effects at 3 or 10 mg/kg (FIG. 2).

### Conclusion:

These results suggest that Compound (I) (1 mg/kg) and Compound (III) (10 mg/kg), administered p.o. acutely significantly decrease the expression of morphine tolerance.

### Example 1b

### Evaluation of the Effects of Mavoglurant on Opiate Dependence Reinstatement Using the Intravenous Self-Administration Model in Sprague-Dawley Rats

Several pre-clinical models have been developed to assess compounds' abuse liability and/or evaluate their efficacy in treating drug dependence. Intravenous self-administration is widely accepted as a valid model to assess the abuse liability of various drugs. For example, using this model, one can assess whether a compound shows efficacy in attenuating opiate self-administration. Using this model, one can also evaluate the efficacy of a compound to prevent relapse after extinction of a conditioned response to opiate.

### Method:

Surgery and Opiate Training: The rats are trained to lever press for food and then are prepared with intravenous catheters. After acquiring the lever-press response, rats are allowed to self-administer an opiate (e.g Morphine, heroin, remifentanil) by pressing the previously food-paired lever in return for delivery of the drug solution (FR1-3, i.e., 1-3 lever-press for each opiate infusion).

### Test Compounds

Mavoglurant (free form) can be formulated in 0.5% methylcellulose (MC) at 1, 3 and 10 mg/kg and administered orally at a dose volume of 1 ml/kg 60 minutes prior to test.

MPEP can be formulated in 0.5% methylcellulose (MC) at 10 mg/kg and administered orally at a dose volume of 1 ml/kg 30 minutes prior to test.

### Methods:

### Phase I: Acute Effects of Mavoglurant on Opiate Self-Administration in Rats

After a stable responding for the training substance is established the vehicle, Mavoglurant (1, 3, 10 mg/kg), MPEP (20 mg/kg i.p. 30 minutes) are evaluated during 1 test session.

### Phase II: Effects of Mavoglurant on Cue-Induced Reinstatement of the Self-Administration Response

The methods for food training, catheter surgery, flushing and infusion confirmation are the same as those in Phase I.

### Extinction and Cue-Induced Reinstatement

After a stable rate of opiate self-administration is achieved, the rats undergo a extinction procedure, which is similar to acquisition training except that pressing the active lever leads to saline infusion instead of the opiate.

Once the extinction is achieved (e.g. significant reduction of active lever presses), rats are pretreated with either the vehicle, test or comparison substances prior to the cue reinstatement session (Tone and cue light).

### Conclusion:

As shown in Example 1a herein above, mavoglurant reduces the tolerance to the analgesic effect of opioid (morphine). The chronic use or misuse of opioid drugs are intimately linked with the development of addiction, dependence and tolerance (Morgan & Christie Brit J Pharm 2011, 164:1322-1334). Tolerance is characterized by a reduced drug efficacy and the need to increase drug dose. Based on this knowledge and the results of Example 1a, it is thus plausible that mavoglurant is efficacious in a model of opiate self-administration and a model of relapse for opiate.

### Example 2

### Evaluation of the Effects of Mavoglurant on Opioid Dependence Using the Remifentanil Intravenous Self-Administration Model in Sprague-Dawley Rats

### Animals

Adult male Sprague-Dawley rats (220-320 g at arrival) from Harlan Laboratory (Indiana, USA) were used. Upon arrival up to the end of the study, rats were housed individually in macrolon cages on wood litter (SAFE, 89290 Augy, France). Additional environmental enrichment (nesting material, gnawing material and tunnel up to the surgery phase) were provided. The animal house was maintained under artificial lighting (12 hours) between 7:00 and 19:00 in a controlled ambient temperature of 22±2° C., a relative humidity between 30-70%.

### Test Compounds

Remifentanil hydrochloride (Ultiva®), (3.2 μg/kg/infusion) was dissolved in saline and administered i.v. at a dose volume of 0.519 mL/kg (0.9% NaCl).

Mavoglurant (free form) was formulated in 0.5% methylcellulose (MC) at 3, 10 and 30 mg/kg and administered orally at a dose volume of 5 ml/kg 30 minutes prior to test.

MPEP Hydrochloride salt [2-methyl-6-(phenylethynyl)-pyridine HCl] was formulated in 0.5% methylcellulose (MC) at 20 mg/kg and administered orally at a dose volume of 5 ml/kg 30 minutes prior to test

Buprenorphine hydrochloride (Buprecare® or Bupaq® Multidose 0.3 mg/mL), diluted in physiological saline and dosed subcutaneously at a dose volume of 5 ml/kg.

### Apparatus

Experiments are conducted in operant chambers (MED Associates, Inc., St. Albans, Vt., U.S.A.) measuring 25.5×29.5×19 cm high and located within sound-attenuating, ventilated cubicles. Each chamber is equipped with a single lever located to the right of a 5×5 cm opening for food pellet delivery from a food hopper. A 2.5 cm translucent stimulus light is located above the food hopper. Experimental events are controlled and monitored, and data are collected and stored, by a computerized system (MED-PCIV, MED Associates, Inc.) and associated interface (MED Associates, Inc.).

### Methods

### Acute Effects of Mavoglurant on Remifentanil Self-Administration in Rats

### Food Training and Surgery

Upon arrival, rats have restricted access to food for 5-7 days (15 g/rat/day then 18 g/rat/day after the surgery), after which lever training begins under a Fixed Ratio 1 (FR1) schedule of food pellets (AIN-76A Rodent Tablet 45 mg, Cambridge University) presentation. Daily sessions comprise 45 minute or 20 food presentations, whichever occurs first. Once rats receive 20 food pellets in a single session under the FR1 schedule (typically within 5 days), food training is suspended and a chronic indwelling i.v. femoral catheter is implanted.

### Acquisition of the Remifentanil Self-Administration Response

At least 3 days after surgery, daily sessions (typical maximum duration of 120 minutes) were conducted during which rats received a maximum of 50 i.v. infusions of the training substance (remifentanil 3.2 μg/kg/infusion) under the fixed ratio (FR1) schedule. Immediately prior to the beginning of daily sessions, rats received 1 non-contingent (i.e., “priming”) infusion of remifentanil 3.2 μg/kg/infusion. A distinctive visual stimulus was displayed during periods when i.v. infusions are available. Each infusion was followed by a 30-second timeout, during which the chamber was kept dark and lever presses had no programmed consequence.

Pharmacological Treatment with Mavoglurant

After responding for the training substance (remifentanil 3.2 μg/kg/infusion) stabilizes (i.e., when the variability in the number of infusions delivered per session does not exceed ±30% over 3 consecutive sessions), the vehicle (p.o. −30 minutes), the three doses of mavoglurant (3, 10 and 30 mg/kg p.o. 30 minutes), the dose of MPEP (20 mg/kg p.o. 30 minutes) and the dose of buprenorphine (0.4 mg/kg s.c. 30 minutes) were evaluated during 1 test session.
N=14 (Total number of rats)
Vehicle (0.5% methylcellulose)
Mavoglurant 3 mg/kg
Mavoglurant 10 mg/kg
Mavoglurant 30 mg/kg
MPEP 20 mg/kg
Buprenorphine 0.4 mg/kg

### Results:

### Effects of Mavoglurant on Remifentanil Self-Administration

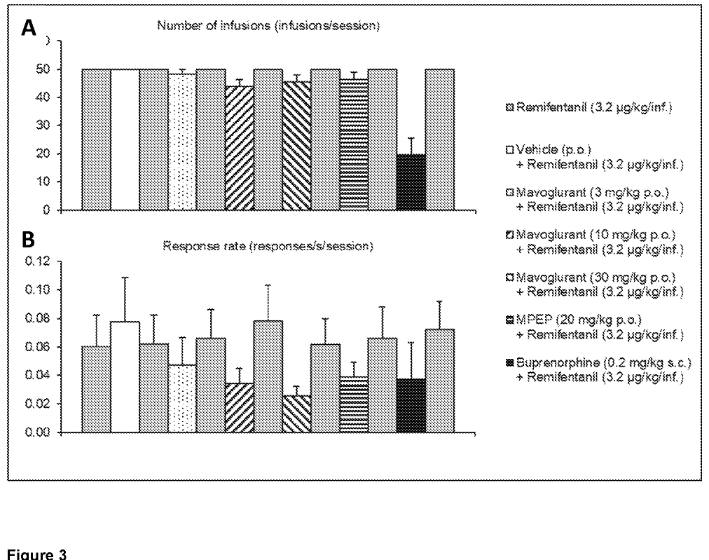

As illustrated on FIG. 3A, Mavoglurant (3, 10, or 30 mg/kg) administered p.o. 30 min before one Remifentanil 3.2 μg/kg/infusion session, did not significantly affect the average number of remifentanil infusions. The rate of lever press (speed of lever pressing reported as number of responses/second/session) was reduced in a dose dependent manner with the treatment with Mavoglurant (3, 10 and 30 mg/kg; 0.048±0.019, 0.035±0.010 and 0.026±0.007, respectively compared to remifentanil 3.2 μg/kg/infusion or the vehicle placebo treatment (0.077±0.031) although not reaching statistical significance (P>0.05) (FIG. 3B).

The mGluR5 antagonist MPEP at 20 mg/kg, administered p.o. 30 minutes before one remifentanil 3.2 μg/kg/infusion session did not significantly affect the average number of remifentanil infusions (FIG. 3A). The rate of lever press was also not significantly reduced (0.039±0.011 responses per second) as compared with the preceding remifentanil 3.2 μg/kg/infusion alone phase or as compared with the vehicle testing session (p>0.05) (FIG. 3B).

The opioid receptor partial agonist Buprenorphine markedly and significantly reduced the average number of remifentanil infusions (FIG. 3A). The rate of lever press was reduced 0.038±0.025 responses per second although not reaching statistical significance (p>0.05, as compared with the preceding remifentanil 3.2 μg/kg/infusion alone phase or as compared with the vehicle testing session) (FIG. 3B).

### Example 3: Evaluation of Mavoglurant in Cue-Induced Reinstatement of Remifentanil Seeking in the Rat

### Food Training and Surgery

The methods for food training, catheter surgery, flushing and infusion confirmation were the same as those in Example 2.

### Acquisition of Remifentanil Self-Administration

Beginning at least 3 days after surgery, daily 2 hour-sessions (Monday to Friday) were conducted during which rats received a maximum of 200 i.v. infusions of the baseline drug (remifentanil, 3.2 μg/kg/infusion) under the FR1 schedule of lever pressing. For each rat only one lever was active. Responding on the inactive lever are recorded but had no programmed consequences. Immediately prior to the beginning of daily sessions, rats received a non-contingent (i.e., “priming”) infusions of remifentanil (3.2 μg/kg/infusion). The house light was on during periods when i.v. infusions were available. During these sessions when rats pressed the active lever, the cue light turned on for 3 sec. After 2 sec of cue light on, a tone signal (2900 Hz sonalert, approx 79 DB) turned on for 1 sec together with activation of the infusion pump. Each infusion was followed by a 30-second timeout, during which the chamber was kept dark and lever presses had no programmed consequence. During the time out period, responses to active lever were recorded but no pump activation/remifentanil infusion occurs.

### Extinction and Cue-Induced Reinstatement

Animals that have reached criterion described above were included into the first extinction phase. Rats were connected to the infusion pump during these sessions but the extinction sessions differ from the self-administration sessions in that active lever presses no longer produce drug infusion or cue light/tone presentation, or noise from infusion pump (pump turned off). These sessions lasted 2 hours and were given daily (Monday to Friday) for a maximum of 15 days. During one of these extinction sessions, rats were pretreated with vehicle (p.o.) to habituate them to drug administration. Rats were considered to have extinguished behavior when during 2 consecutive sessions they exhibited an average of fewer than 12 active lever presses or a maximum of 25% of the number of active lever presses that occurred during the last 2 sessions of remifentanil self-administration, whichever comes first.

First cue-induced reinstatement: On the next working day, rats are pretreated with either the vehicle, Mavoglurant (3 and 10 mg/kg), MPEP (20 mg/kg/) p.o. 30 minutes before the cue reinstatement session. The cue reinstatement session begins with a tone (1 sec) and cue light (3 sec). The remainder of the 2 hours session is identical to the self-administration session (cue light and tone present upon active lever press) except that no remifentanil was delivered and that there was no 30-second timeout. Lever presses were recorded.

Second extinction: Rats were submitted to a second extinction period for a maximum of 15 days. Similarly to the first extinction period, rats are connected to the infusion pump during these sessions but the extinction sessions differ from the self-administration sessions in that active lever presses no longer produce drug infusion or cue light/tone presentation, or noise from infusion pump (pump turned off).

Second cue-induced reinstatement: Similarly to the first Cue-induced reinstatement session, rats were pretreated with either the vehicle, Mavoglurant (3 and 10 mg/kg), MPEP (20 mg/kg/) p.o. 30 minutes before the cue reinstatement session. The cue reinstatement session began with a tone (1 sec) and cue light (3 sec). The remainder of the 2 hours session was identical to the self-administration session (cue light and tone present upon active lever press) except that no remifentanil was delivered and that there was no 30-second timeout. Lever presses were recorded. The number of active lever presses obtained during the reinstatements sessions was compared to extinction lever responding. This was considered a measure of reinstatement.

### Results:

### Effects of Mavoglurant on Cue-Induced Reinstatement of Self-Administration

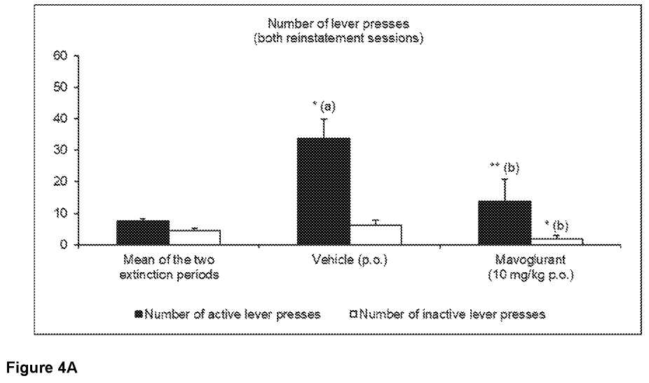

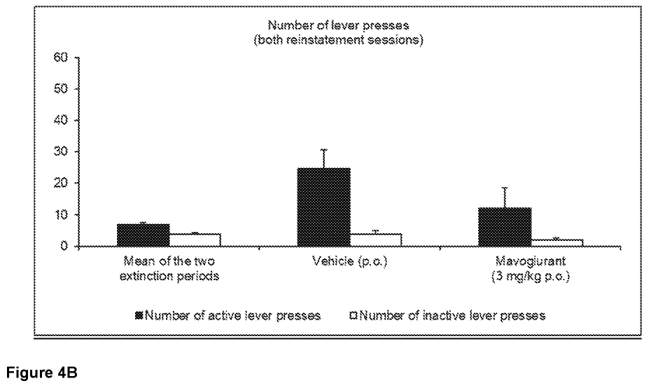

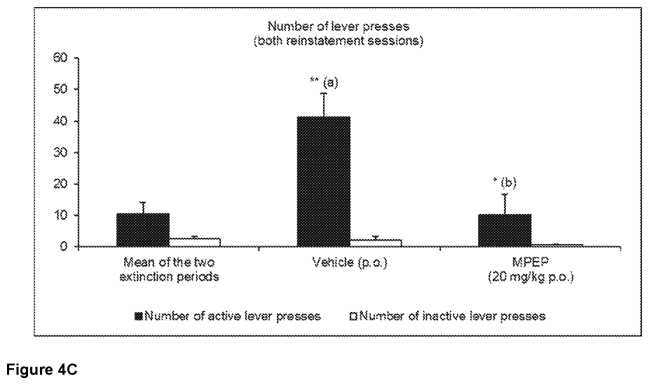

MPEP at 20 mg/kg significantly decreased the number of active lever presses as compared with vehicle condition (p<0.05) but did not significantly affect the number of inactive lever presses as compared with vehicle (FIG. 4C).

Mavoglurant at 10 mg/kg significantly decreased the number of active lever presses as compared with vehicle condition (p<0.01). In the same manner, Mavoglurant at 10 mg/kg, significantly decreased the number of inactive lever presses as compared with vehicle (p<0.05) (FIG. 4A). No significant effect was observed in group treated with Mavoglurant at 3 mg/kg p.o. (FIG. 4B).

The present study showed that Mavoglurant administrated at 10 mg/kg is effective in reducing the cue-induced relapse to the self-administration of remifentanil. Based on these results it is thus plausible that mavoglurant attenuates the relapse into opioid use by an opioid use disorder patient.

### Study Objectives and Endpoints

Primary Objective(s)
To evaluate treatment effect of 98-day mavoglurant administration in reducing opioid use

Secondary Objective(s)
To assess the effects of 98-day mavoglurant administration versus placebo on:
a) other measures of opioid use
b) alcohol use
To assess the safety and tolerability of multiple bid oral doses of mavoglurant
To evaluate the pharmacokinetics of mavoglurant

### Exploratory Objective(s)

To examine whether individual genetic variation in genes relating to drug metabolism and transporters, OUD and the drug target pathway confer differential response to mavoglurant
To assess the effects of 98-day mavoglurant administration versus placebo on:
a) depressive symptoms
b) anxiety symptoms
c) global functioning

Endpoints Related to Primary Objective(s)
Proportion of opioid use days by TLFB opioid self-report

Endpoints Related to Secondary Objective(s)
Urinalysis [e.g. by measuring metabolites of opioids in urine]
b) TLFB alcohol self-report; urinalysis [Ethyl Glucuronide (EtG)]
Vital signs, ECG parameters, clinical safety laboratory parameters (chemistry/hematology/urinalysis), (serious) adverse events reporting, suicidal ideation [Columbia Suicide Severity Rating Scale (C-SSRS)]
mavoglurant plasma concentrations (pre- and post-dose levels)

### Endpoints Related to Exploratory Objective(s)

genetic markers in pharmacogenetic or biologic candidate genes for OUD, target or drug response
a) Beck's Depression Inventory (BDI)
b) State-Trait Anxiety Inventory (STAI)
c) Clinical Global Impression Scale—Severity (CGI-S) and Improvement (CGI-I)

### Study Design

This is a randomized, subject and investigator blinded, parallel group, placebo-controlled study in patients with OUD. The study consists of: about 21-day screening period followed by a 7-day baseline; a 98-day out-patient treatment period (14-day up-titration dose regimen followed by 84-day maintenance dose) and finally an end of study visit evaluation approximately 14-days after the last study drug administration. The total duration for each patient in the study is up to approximately 20-weeks.

Study visits (days 1-112): study visits are performed in an ambulatory setting of frequency twice per week. During these visits the urine samples are collected for drug screen (opioid and others drug metabolites) along with safety/efficacy assessments.

Screening (days −28 to −8): includes safety examinations and other clinical tests, determines patients' initial eligibility. Patients who meet the eligibility criteria at screening are admitted to the baseline evaluation.

Baseline (days −7 to −1): includes, in addition to the safety evaluations, a patient's self-assessment on various scales and questionnaires. During baseline, a history of self-reported opioid use (TLFB) and two urine samples are collected on two different days with second sample collected 3 days prior randomization to demonstrate the abstinence from opioid use.

Treatment (days 1-98): following baseline, on Day 1, eligible patients are randomly assigned in a 1:1 ratio to either mavoglurant (free form) or placebo
Group A—mavoglurant (free form): up-titration regimen for the first 2 weeks: 50 mg bid from Day 1 to Day 7, 100 mg bid from Day 8 to Day 14, followed by dosing at 200 mg bid for 84-days
The dose selected (200 mg b.i.d/modified release formulation) for evaluation in this study has been chosen based on the safety, tolerability and pharmacokinetic data from completed mavoglurant studies.
Group B: matching placebo.

Study drug: taken twice daily (b.i.d.), in the morning and evening (separated by approximately 12 hour intervals), with food. For all ambulatory morning visits that involve any study assessments or PK/urine sample collection, study medication is self-administered by patient at study center and supervised by study personnel. On these days, standard breakfast is served at study center and consumed by the patient during his/her medication intake.

During treatment period, patients also undergo assessments with various scales and questionnaires, as well as safety assessments and pharmacokinetic sampling at pre- and 2±1-hour post dose per SoA.

Urine samples (days 1-113): samples are collected at study center twice per week, with at least 48 hours separating tests, e.g. Tuesday and Fridays or Mondays and Thursdays or Tuesday morning and Thursday afternoon. The sample collection is staff-observed and is assayed quantitatively for the presence of opioid and alcohol metabolites [e.g. Ethyl Glucuronide (EtG)]. Urine samples are collected during study conduct: 28 samples from patients who remain in treatment for the 14 weeks (4 samples in weeks 1-2 of up-titration); 24 samples in weeks 3-14 (maintenance dose); 4 samples in weeks 15-16 (follow up) and finally last 1 sample at end of study visit. If a patient fails to attend the clinic or refuses to provide a sample on a scheduled testing day, samples are considered positive unless an excused absence is granted (e.g. illness, other personal reason). In cases of missed or refused samples, samples are collected on the next day whenever possible.

### Clinical Support to Ensure Medication and Protocol's Adherence:

Medication compliance: patients are at the study site at time of study drug administration for the morning dose on PK collection days and on all other days that involves urine sampling assessments. On these days study medication self-administration is supervised by study personnel, compliance is ensured by mouth check after the medication is swallowed. To monitor medication adherence, patients are provided with individual Medication Diary (booklet) to record administration of study medication. Medication compliance is monitored by the investigator and/or study personnel at least on a weekly basis using tablet(s) counts. Dosage adherence is verified by comparing the patient's Medication Diary self-reported data against the total number of tablets in the returned bottle or blister (depends on the packaging form). Adherence is calculated as the total amount of tablets taken divided by the scheduled total amount to be taken during the treatment phase. If the Investigator feels it is appropriate, the patient may also be contacted during the out-patient periods to confirm compliance.

### Population

Patients, aged of 18-65 inclusive with a diagnosis of OUD, who use opioid, are enrolled in the study.

### Statistical Model and Method of Analysis

The primary variable: is the proportion of opioid use days during the treatment period (days 1-98).

For each patient, the proportion of opioid use days is calculated by dividing the number of days of opioid use during the treatment period, i.e. 98 days for completers and number of days between Day 1 and day of last dose in case of premature discontinuation of study treatment. It is considered as a continuous variable. The opioid consumption is recorded daily (Yes/No) using the TLFB during the entire study.

A Bayesian analysis is conducted on the proportion of opioid use days. It is assumed to be a continuous outcome with normally distributed errors. A linear model with treatment factor and past consumption of opioid as covariate is fitted. Non informative priors are used for all parameters. The past consumption of opioid is the proportion of opioid use days over the 3 months preceding the screening visit, which is assessed using the TLFB. The model evaluates the posterior probability that the difference in the proportions of opioid use days between mavoglurant and placebo is <0 and that its <−10%. The difference of 10% of days is deemed the minimal clinically relevant effect and a difference of 20% is a very promising effect, based on the literature in this indication. The study shows a positive signal of efficacy if the 2 following criteria are met:
1. there is at least 90% probability that the difference in the proportions of opioid use days between mavoglurant and placebo is <0
2. there is at least 50% probability that the difference in the proportions of opioid use days between mavoglurant and placebo is <−10%

Sensitivity analyses are performed using other models, like negative binomial regression if the distribution of the data is not normal. Additionally, the profiles of consumption over time are compared between treatment groups through analyses of longitudinal data (weekly use) using mixed models for repeated measures.

The secondary variables: include the proportion of negative UDS over the treatment period and will be analyzed in the same way as the primary variable. Safety and PK are secondary endpoints for this study.

1. Use of mavoglurant, or a pharmaceutically acceptable salt thereof, for the manufacture of a medicament for a treatment in the reduction of opioid use by an opioid use disorder patient.

2. Use of mavoglurant, or a pharmaceutically acceptable salt thereof, for the manufacture of a medicament for a treatment to prevent relapse into opioid use by an opioid use disorder patient.

3. Use of mavoglurant, or a pharmaceutically acceptable salt thereof, for the manufacture of a medicament for a treatment to promote opioid abstinence by an opioid use disorder patient.

4. Use of mavoglurant, or a pharmaceutically acceptable salt thereof, for the manufacture of a medicament for the treatment of the symptoms of depression or anxiety associated with opioid use disorder.

5. Use of mavoglurant, or a pharmaceutically acceptable salt thereof, for the manufacture of a medicament for the treatment according to any one of claims 1 to 4, wherein opioid use disorder is associated with opioid inhalation (i.e. smoking), intravenous opioid, opioid nasal insufflation (i.e. snorting) or oral formulations of opioid; in particular is associated with opioid inhalation (i.e. smoking).

6. Use of mavoglurant, or a pharmaceutically acceptable salt thereof, for the manufacture of a medicament for the treatment according to any one of claims 1 to 5, wherein opioid use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.

7. Use of mavoglurant, or a pharmaceutically acceptable salt thereof, for the manufacture of a medicament for the treatment according to any one of claims 1 to 6, wherein the use is combined with standardized psychological treatment, for example, at individual or group level.

8. Use of mavoglurant, or a pharmaceutically acceptable salt thereof, for the manufacture of a medicament for the treatment according to any one of claims 1 to 7, wherein the use is combined with psychosocial or behavioral therapy or combination thereof, in particular contingency management based therapy.

9. Use of mavoglurant, or a pharmaceutically acceptable salt thereof, for the manufacture of a medicament for the treatment according to claim 8, wherein the psychosocial or the behavioral therapy is computer-assisted.

10. Use of mavoglurant, or a pharmaceutically acceptable salt thereof, for the manufacture of a medicament for the treatment according to any one of claims 1 to 9, wherein the use is concomitant with methadone or buprenorphine treatment.

11. Use of mavoglurant, or a pharmaceutically acceptable salt thereof, for the manufacture of a medicament for the treatment according to any one of claims 1 to 10, wherein mavoglurant, or a pharmaceutically acceptable salt thereof, is administered in combination with a further active agent.

12. Use of mavoglurant, or a pharmaceutically acceptable salt thereof, for the manufacture of a medicament for the treatment according to any one of claims 1 to 11, wherein the patient has a genetic variation associated with a substance use disorder, such as a genetic variation associated with opioid use disorder.

13. Use of mavoglurant, or a pharmaceutically acceptable salt thereof, for the manufacture of a medicament for the treatment according to any one of claims 1 to 12, wherein mavoglurant, or a pharmaceutically acceptable salt thereof, is administered in an immediate-release form or a modified-release form.

14. Use of mavoglurant, or a pharmaceutically acceptable salt thereof, for the manufacture of a medicament for the treatment according to any one of claims 1 to 13, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.

15. Use of mavoglurant, or a pharmaceutically acceptable salt thereof, for the manufacture of a medicament for the treatment according to any of the preceding claims, wherein opioid use disorder is associated with binge drinking.

16. Use of mavoglurant, or a pharmaceutically acceptable salt thereof, for the manufacture of a medicament for the treatment of opioid use disorder.
